(19) United States
(12) Patent Application Publication
(10) Pub. No.: US 2026/0062754 A1
(43) Pub. Date: Mar. 5, 2026

(54) COMPOSITION FOR PREVENTING OR TREATING DIABETIC CARDIOMYOPATHY COMPRISING CONTAINING A MSRB2 ACTIVATOR AS AN ACTIVE INGREDIENT

(71) Applicant: Korea National Institute of Health, Cheongju-si (KR)

(72) Inventors: Seung Hee LEE, Cheongju-si (KR); Su Yeon CHO, Cheongju-si (KR); Won-Ho KIM, Cheongju-si (KR)

(73) Assignee: Korea National Institute of Health, Cheongju-si (KR)

(21) Appl. No.: 19/310,760

(22) Filed: Aug. 26, 2025

(30) Foreign Application Priority Data
Aug. 29, 2024 (KR) 10-2024-0116995

(51) Int. Cl. C12Q 1/6883; G01N 33/50; G01N 33/573

(52) U.S. Cl. CPC C12Q 1/6883; G01N 33/5023; G01N 33/573; C12Q 2600/158; G01N 2333/90212

(57) ABSTRACT

The present invention relates to a composition for preventing or treating diabetic cardiomyopathy comprising an MsrB2 activator as an active ingredient. The MsrB2 activator disclosed in the present invention suppresses oxidative damage of ROS, induces autophagy in mitophagy, increases mitochondrial regeneration and biosynthesis in diabetic heart tissue, thereby suppressing overall myocardial function decline, so that it can be effectively used in a pharmaceutical composition or health food composition for preventing or treating diabetic cardiomyopathy, particularly diabetic heart disease in lean diabetes.

### CROSS-REFERENCE TO RELATED APPLICATIONS

This application claims the benefit of priority under the Paris Convention from Korean Patent Application No. 10-2024-0116995, filed Aug. 29, 2024. The contents of the above application are hereby incorporated by reference in their entirety.

### BACKGROUND OF THE INVENTION

### Field of the Invention

The present invention relates to a composition for preventing or treating diabetic cardiomyopathy comprising an MsrB2 activator as an active ingredient. The present invention provides a composition that regulates mitochondrial biogenesis and improves cardiac function by reducing ROS production and modulating MsrB2 expression in cardiac tissue.

Diabetes mellitus (DM) is a progressive, chronic metabolic disorder characterized by hyperglycemia due to impaired insulin levels, sensitivity, or action. Diabetes is the 6th leading cause of death in Korea, 16.7% of adults over the age of 30 have diabetes, and 44.3% are in the pre-diabetic stage. The diabetes awareness rate is 65.8%, and the treatment rate is 61.4%, but the control rate is only 24.5%.

Diabetes can be classified into four types based on the cause: type 1 diabetes, type 2 diabetes, gestational diabetes, and diabetes caused by another condition, such as a genetic mutation. Among these, type 2 diabetes, which accounts for the majority of diabetes, has various forms, from cases in which insulin secretion deficiency progresses early after onset to cases in which insulin resistance due to obesity is compensated for by excessive insulin secretion. In addition, type 2 diabetes in Korea is known to develop at an earlier age, cause early complications, and shorten average life expectancy, and more than 50% of diabetic patients are thin.

Diabetes is an important disease that requires careful management, as it is well known not only as a problem of hyperglycemia itself, but also as a direct and indirect cause of various chronic complications, including cardiovascular and cerebrovascular diseases. In particular, in many Asian countries, including Korea, the number of diabetes patients is increasing at an explosive rate greater than in Western societies. Unlike Westerners, the rate of systemic obesity in Korean type 2 diabetes patients is 53.2% of the diabetic population, and there are many thin diabetic patients, so it is necessary to develop prevention and management indicators that can effectively manage them and prevent complications from occurring. Due to type 2 diabetes, 28.6% of diabetic patients have macrovascular complications such as peripheral arterial disease, and 67.2% have microvascular complications such as nephropathy, and the annual medical expenditure for diabetes in Korea is about 3 trillion won.

It is known that 65% of people with diabetes die from thrombotic cardiovascular diseases, including heart attacks and strokes. Heart failure in diabetes (diabetic cardiomyopathy) increases with factors such as atherosclerosis, hypertension, and myocardial dysfunction. Myocardial stiffness due to matrix protein accumulation (cardiac fibrosis) contributes to the development of diabetic cardiomyopathy (DCM). However, other pathophysiologic mechanisms such as myocardial insulin resistance, reactive oxygen species (ROS), and inflammation have been implicated in the development of diabetic cardiomyopathy. Conversely, mitophagy can ameliorate cardiac dysfunction in diabetic cardiomyopathy, but the exact molecular mechanisms of protection conferred by mitophagy remain unclear.

Diabetic cardiomyopathy is caused by abnormal Ca²⁺-induced myofibrillar ATPase activity and changes in myofibrillar network function. SERCA2a is critical in the pathogenesis of diabetic cardiomyopathy. Overexpression of SERCA2a improves mitochondrial quality control and attenuates cardiac microvascular ischemia-reperfusion injury.

Damaged mitochondria significantly increase reactive oxygen species (ROS) levels, which can alter various protein functions through post-translational modifications such as carbonylation, 3-nitrotyrosine, s-sulfonation, s-nitrosylation, s-glutathionylation, disulfide formation, methionine oxidation (MetO), and ubiquitination. In addition, ROS can increase mitophagy as a protective mechanism to remove damaged mitochondria and prevent their overproduction. However, the molecular mechanism between myocardial ROS and diabetic cardiomyopathy has not yet been elucidated.

MsrB2 is known to be a mitophagy inducer in diabetic platelets. In platelets, MsrB2 plays a key role in regulating damaged mitochondria to be controlled by mitophagy. However, the function of MsrB2 in other tissues in diabetes is unknown.

As a prior art of the present invention, there is U.S. Pat. No. 10,519,502B2 (Genetic Diagnosis of Mitochondrial Diseases), which discloses that the MsrB2 gene was used to diagnose a mitochondrial genetic disease. However, this is for the diagnosis of a mitochondrial genetic disease, and Lee et al. (EMBO Mol Med. 2019) reported that the expression of MsrB2 increased in platelets of a diabetic complication patient, but only mentioned a thrombotic disease as a diabetic complication, which is different from the diabetic cardiomyopathy of the present invention. In addition, Yi Li et al. (Experimental Eye Research, 2011) confirmed the effect of early-stage diabetes on the methionine sulfoxide reductase (Msrs) expression, and mentioned the association between diabetes and the Msr mRNA expression, but did not mention diabetic cardiomyopathy.

The present inventors confirmed that the MsrB2 gene expression is increased in the diabetic heart of mice and humans, and that an MsrB2 activator targeting this gene can be useful in preventing, treating, and ameliorating heart disease heart disease caused by type 2 diabetes by suppressing oxidative damage of ROS, inducing autophagy in mitophagy, activating mitochondrial regeneration and biosynthesis in diabetic heart tissue, and suppressing myocardial damage, and particularly suggesting that it is effective in lean type 2 diabetes, thereby completing the present invention.

### SUMMARY OF THE INVENTION

It is an object of the present invention to provide a pharmaceutical composition for preventing or treating diabetic cardiomyopathy comprising an MsrB2 activator as an active ingredient.

It is another object of the present invention to provide a health functional food for preventing or ameliorating diabetic cardiomyopathy comprising an MsrB2 activator as an active ingredient.

It is another object of the present invention to provide a screening method of an effective substance having preventive or therapeutic activity against diabetic cardiomyopathy.

To achieve the above objects, the present invention provides a pharmaceutical composition for preventing or treating diabetic cardiomyopathy comprising an MsrB2 activator as an active ingredient.

The present invention also provides a health functional food for preventing or ameliorating diabetic cardiomyopathy comprising an MsrB2 activator as an active ingredient.

In addition, the present invention provides a method for screening an effective substance having preventive or therapeutic activity against diabetic cardiomyopathy.

### Advantageous Effect

A composition comprising the MsrB2 activator of the present invention as an active ingredient can provide the effect of inhibiting oxidative damage by activating LC3II and Parkin, inducing autophagy in mitophagy, activating energy metabolism by reducing ROS production, maintaining myocardial contractility in cardiac tissue by increasing the expression of SERCA2a and phospholamban, and suppressing overall myocardial function decline by increasing mitochondrial regeneration and biosynthesis, and thus can be effectively used as a pharmaceutical composition for preventing or treating diabetes or a health functional food composition for preventing or ameliorating diabetes, particularly diabetic cardiomyopathy in lean diabetes.

### BRIEF DESCRIPTION OF THE DRAWINGS

FIG. 1a is a diagram showing the results of comparing the blood sugar levels in diabetic mice (DM) and non-diabetic mice (non-DM).

FIG. 1b is a diagram showing the results of Western blotting of MetO and MsrB2 in the hearts of non-diabetic mice (#1-3) and diabetic mice (DM #1-3). GAPDH was used as a loading control.

FIG. 1c is a diagram showing the quantification of the signal intensities of MetO and MsrB2 in diabetic mice (DM) and non-diabetic mice (non-DM).

FIG. 1d is a diagram showing the results of Western blotting of Parkin and LC3II in the hearts of non-diabetic mice (#1-3) and diabetic mice (DM #1-3). GAPDH was used as a loading control.

FIG. 1e is a diagram showing the quantification of the signal intensities of Parkin and LC3II in diabetic mice (DM) and non-diabetic mice (non-DM).

FIG. 1f is a diagram showing the results of Western blotting of MsrB2 and LC3I/II in the hearts of non-diabetic mice (#1-3), diabetic mice (DM #1-3), and NAC-treated DM (#1-5). GAPDH was used as a loading control.

FIG. 1g is a diagram showing the quantification of the signal intensities of MsrB2 and LC3I/II in diabetic mice (DM), non-diabetic mice (non-DM), and NAC-treated DM (#1-5).

FIG. 1h is a diagram showing the results of Western blotting of LC3I/II of H9C2 under the 5.5 mM and 25 mM glucose treatment conditions.

FIG. 1i is a diagram showing the results of observing H9C2 cells cultured with 1 uM H2DCFDA for 1 hour.

FIG. 1j is a diagram showing the quantification (n=3) of the green fluorescence signal intensity of FIG. 1i.

FIG. 1k is a diagram showing the results of observing H9C2 cells treated with ET-1 and incubated with 1 uM H2DCFDA for 1 hour.

FIG. 1l is a diagram showing the quantification (n=3) of the green fluorescence signal intensity of FIG. 1k.

FIG. 1m is a diagram showing the results of Western blotting of MsrB2 and LC31/II of H9C2 under the 5.5 mM and 25 mM glucose treatment conditions.

FIG. 1n is a diagram showing the quantification (n=3) of the signal intensities of MsrB2 and LC3II.

FIG. 2a is a diagram showing the results of electron microscopy (EM) observation of non-diabetic mice. The white body represents mitophagy (autophagosome and autolysosome) structures.

FIG. 2b is a diagram showing the results of electron microscopy observation of diabetic mice.

FIG. 2c is a diagram showing the quantification of mitochondrial size in non-diabetic and diabetic mice.

FIG. 2d is a diagram showing the results of Western blotting of DRP1, OPA1 and Mfn2 in the hearts of non-diabetic mice (non-DM, #1-3) and diabetic mice (DM, #1-3). GAPDH was used as a loading control.

FIG. 2e is a diagram showing the quantification of the signal intensity of DRP1 in diabetic mice (DM) and non-diabetic mice (non-DM).

FIG. 3a is a diagram showing the tissue distribution of MsrB2 in mice, and showing the results of Western blotting of MsrB2 in the heart, muscle, liver, and brain of nondiabetic and diabetic mice. GAPDH was used as a loading control.

FIG. 3b is a diagram showing the results of Western blotting of MsrB2 in the heart, aorta, and liver of wild-type (WT) and knockout (KO) mice. Actin was used as a loading control.

FIG. 3c is a diagram showing the results of a glucose tolerance test (GTT) performed on wild-type (WT) and MsrB2 knockout (KO) mice under the non-diabetic and diabetic conditions.

FIG. 3d is a diagram showing the body weights of WT and MsrB2 KO DM mice fed a high-fat diet (HFD). Body weight was measured weekly.

FIG. 3e is a diagram showing the results of electron microscopy observation of WT and MsrB2 KO DM mice. White arrows indicate mitochondria, black triangles indicate membrane structures, and white triangles indicate abnormal Z-lines.

FIG. 3f is a diagram showing the results of H&E and trichrome staining of heart tissue sections of wild-type and MsrB2 KO mice under the non-diabetic and diabetic conditions.

FIG. 3g is a diagram showing the results of quantifying the fibrotic area in the tissue section using the ImageJ (FIJI) analyzer.

FIGS. 4a to 4i are diagrams showing the results of electrocardiogram measurements of the hearts of non-diabetic (WT #1-4, MsrB2 KO #1-5) and diabetic mice (WT #1-11, MsrB2 KO #1-9).

FIG. 5a is a diagram showing the results of Western blotting of MsrB2, LC3I/II, Parkin, SERCA2a, pPNL, PNL and GAPDH in the hearts of non-diabetic mice (WT #1-3, MsrB2 KO #1-3) and diabetic mice (WT #1-3, MsrB2 KO #1-6). GAPDH was used as a loading control.

FIGS. 5b to 5d are diagrams showing the quantification of the signal intensities of MsrB2, LC3I/II, Parkin, SERCA2a, pPNL/PNL and GAPDH.

FIG. 5e is a diagram showing the results of quantitative analysis of the expression levels of MsrB2 and SERCA2a in the hearts of non-diabetic (WT #1-5, MsrB2 KO #1-5) and diabetic mice (WT #1-3, MsrB2 KO #1-6) by RT-PCR.

FIG. 6a and FIG. 6b are diagrams showing the results of quantitative RT-PCR of the expression levels of NOX4 and SOD1, the ROS inducers, in the mouse heart.

FIG. 6c is a diagram showing the results of quantitative RT-PCR of the expression level of mND1, an OXPHOS-related gene, in the mouse heart.

FIG. 6d is a diagram showing the results of quantitative RT-PCR of the mitochondrial content (mtDNA) levels in the mouse heart.

FIG. 7a is a diagram showing the results of Western blotting of MsrB2 and LC3I/II of H9C2 after MsrB2-GFP transfection. GAPDH was used as a loading control.

FIG. 7b is a diagram showing the quantification of the signal intensities of MsrB2 and LC3II of FIG. 7a.

FIG. 7c is a diagram showing the results of Western blotting of MsrB2, SERCA2a, and bMHC in WT and MsrB2 KO NMCM. GAPDH was used as a loading control.

FIG. 7d is a diagram showing the quantification of the signal intensities of SERCA2a and bMHC of FIG. 7c.

FIG. 7e is a diagram showing the results of Western blotting of MsrB2, LC3I/II, pDRP1 and DRP1 in NMCM after Ad-MsrB2-GFP transfection. GAPDH was used as a loading control.

FIG. 7f is a diagram showing the quantification of the signal intensities of LC3II and pDRP1 of FIG. 7e.

FIG. 8a is a diagram showing the results of Western blotting of MetO in normal (NH #1-3) and diabetic human heart tissues (DH #1-6). GAPDH was used as a loading control.

FIG. 8b is a diagram showing the quantification of the signal intensity of MetO of FIG. 8a.

FIG. 8c is a diagram showing the results of Western blotting of methionine sulfoxide reductase A and B2 (MsrA and B2) in normal (NH #1-3) and diabetic heart tissues (DH #1-6). GAPDH was used as a loading control.

FIG. 8d is a diagram showing the quantification of the signal intensities of MsrA and MsrB2 of FIG. 8c.

FIG. 8e is a diagram showing the results of Western blotting of LC3I/II and p62 in normal (NH #1-3) and diabetic heart tissues (DH #1-6). GAPDH was used as a loading control.

FIG. 8f is a diagram showing the quantification of the signal intensities of LC3I/II and p62 of FIG. 8e.

FIG. 8g is a diagram showing the results of Western blotting of PINK and Parkin in normal (NH #1-3) and diabetic human heart tissues (DH #1-6) to confirm the level of mitophagy-related expression in human diabetic hearts. GAPDH was used as a loading control.

FIG. 8h is a diagram showing the quantification of the signal intensities of PINK and Parkin of FIG. 8g.

FIG. 8i is a diagram showing the results of Western blotting of DRP1 and OPA1 in normal (NH #1-3) and diabetic heart tissues (DH #1-6). GAPDH was used as a loading control.

FIG. 8j is a diagram showing the quantification of the signal intensities of DRP1 and OPA1 of FIG. 8i.

FIG. 9 is a schematic diagram showing a summary of the present, wherein it was confirmed that when MsrB2 expression increases in the heart of a diabetic mouse, cardiac complications are suppressed

### DETAILED DESCRIPTION OF THE INVENTION

Hereinafter, the present invention is described in detail.

The present invention provides a pharmaceutical composition for preventing or treating diabetic cardiomyopathy comprising an MsrB2 activator as an active ingredient.

In the present invention, the MsrB2 activator may be one that activates or upregulates the MsrB2 gene expression.

In the present invention, the MsrB2 activator may be a synthetic or natural compound capable of activating or upregulating the MsrB2 gene expression.

In the present invention, the MsrB2 activator may be a synthetic or natural compound capable of increasing the MsrB2 protein expression and/or activity.

For example, the MsrB2 activator of the present invention may be selected through a screening method according to the present invention, targeting known synthetic compounds or natural compounds.

In the present invention, the diabetic cardiomyopathy may be diabetic cardiomyopathy in a lean diabetic patient.

The composition may inhibit ROS production by increasing the expression and/or activity of MsrB2.

The composition of the present invention activates LC3II and Parkin.

The composition of the present invention activates energy metabolism by inhibiting ROS production.

The composition of the present invention improves cardiac function by increasing the expression of SERCA2a and phospholamban and maintaining myocardial contractility in cardiac tissue.

When the pharmaceutical composition of the present invention is formulated, it may include carriers, diluents, excipients, or combinations of two or more thereof commonly used in pharmaceutical compositions. The pharmaceutically acceptable carriers are not particularly limited as long as they are suitable for delivering the composition into a living body.

The pharmaceutical composition of the present invention can be formulated as oral preparations or parenteral preparations.

The composition of the present invention may include an active ingredient in any amount (effective amount) as long as it can exhibit the intended activity of ameliorating muscle strength loss due to aging, improving muscle loss due to aging, etc., depending on the specific use, formulation, product form, etc. A typical effective amount of the active ingredient may be determined within a range of 0.001 wt % to 15 wt % based on the total weight of the composition. Herein, the “effective amount” refers to the amount of an active ingredient included in the composition of the present invention that can exhibit the intended medical and pharmacological effects, such as the effect of ameliorating muscle strength loss due to aging, when the composition of the present invention is administered to a mammal, preferably a human, which is the subject of application, for an administration period as recommended by a medical professional or the like. Such effective amounts can be determined experimentally within the normal capabilities of those skilled in the art.

The present invention provides a health functional food composition for preventing or ameliorating diabetic cardiomyopathy comprising an MsrB2 activator as an active ingredient.

The term “health functional food” in this specification refers to a food product manufactured using nutrients that are easily deficient in daily meals or raw materials or ingredients that have functions useful to the human body, and is used to mean, but is not limited to, food that helps maintain human health, and includes all health foods in the conventional sense.

The form and type of the above health functional food are not particularly limited. Specifically, the health functional food may be in the form of tablets, capsules, powders, granules, liquids, and pills.

The present invention provides a screening method for an effective substance having preventive or therapeutic activity against diabetic cardiomyopathy, comprising the following steps:
1) a step of preparing cardiomyocytes isolated from a subject;
2) a step of measuring the ROS activity and MsrB2mRNA expression levels before and after treating the prepared cardiomyocytes with candidate substances; and
3) a step of determining a candidate substance that increases the MsrB2 mRNA expression and reduces the ROS activity compared to before treatment with the candidate substance, as an effective substance with therapeutic activity for diabetic cardiomyopathy.

In the present invention, the subject may include, without limitation, mammals, including mice, livestock, humans, and the like, in which the diabetic cardiomyopathy disease is likely to develop or has developed.

In the present invention, the cardiomyocytes may be mouse cardiomyocytes (H9C2), preferably cardiomyocytes isolated from the heart of a mouse born within 3 days.

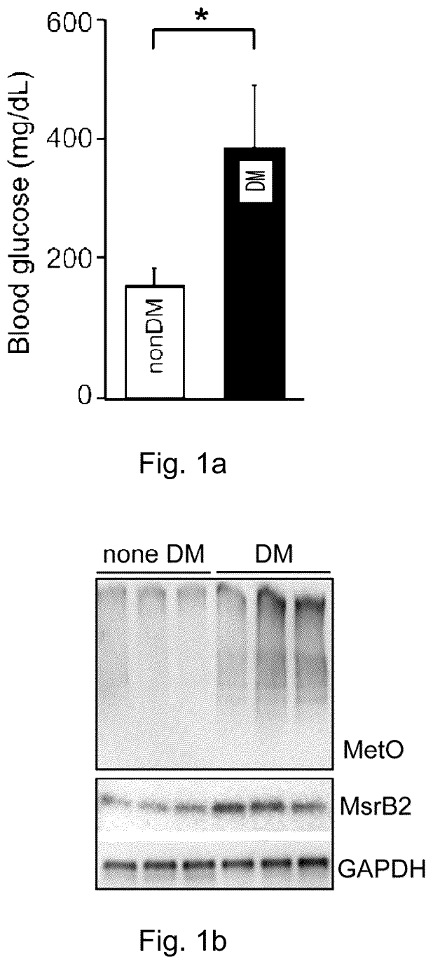

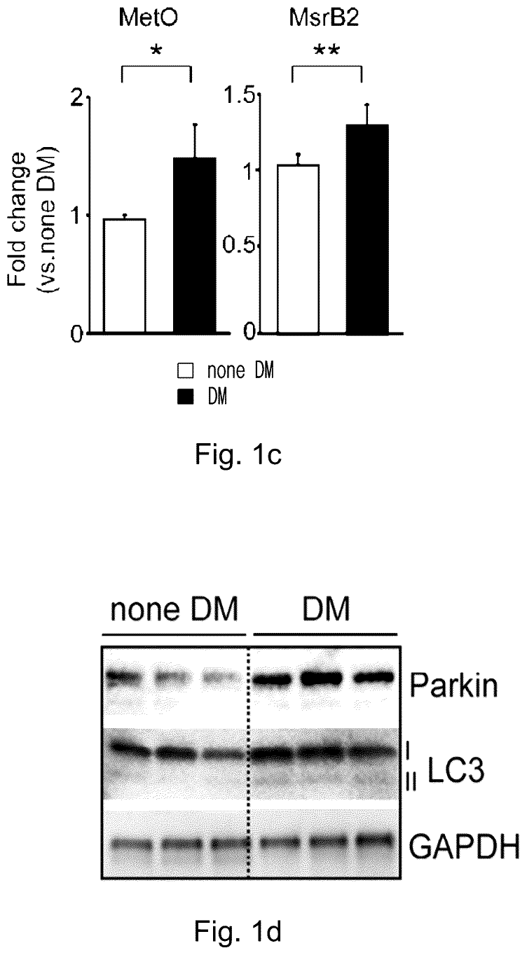

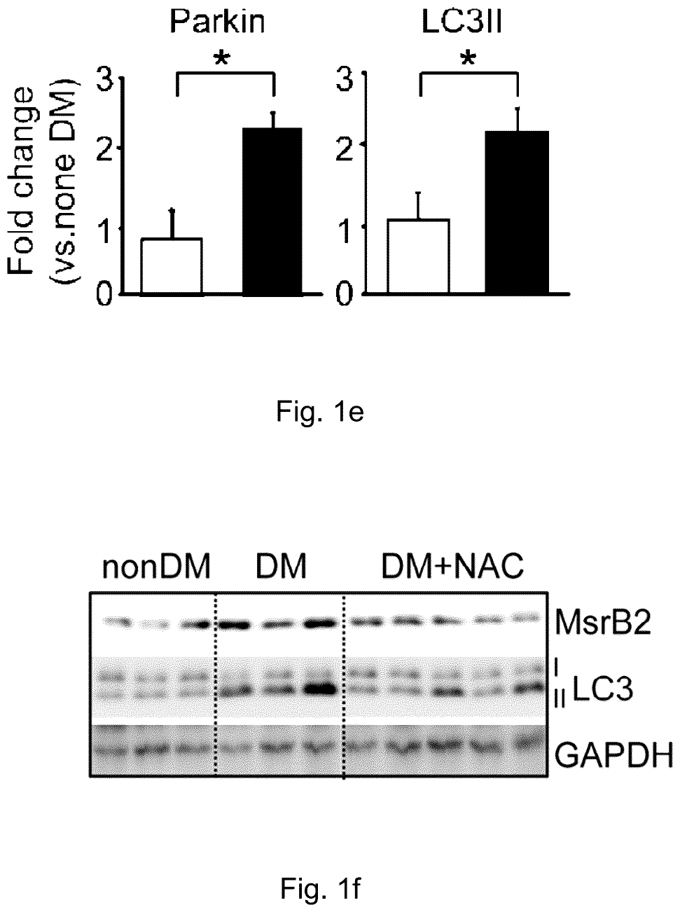

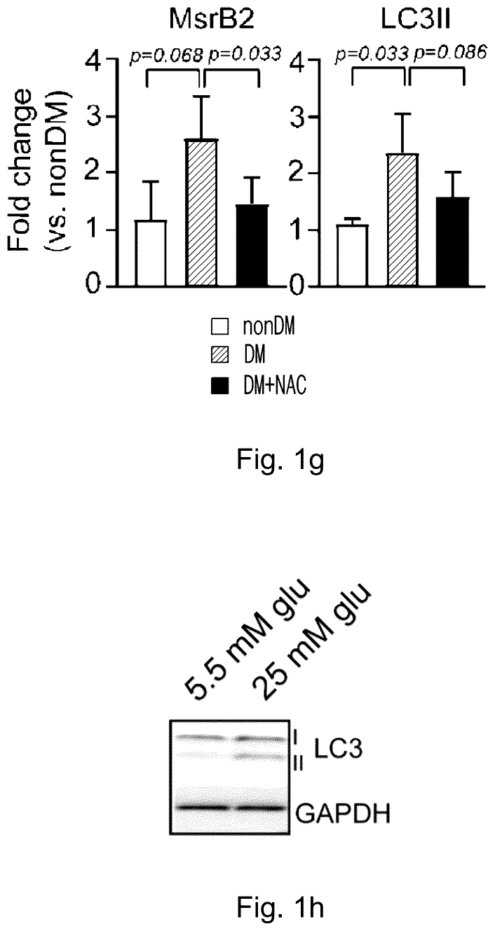

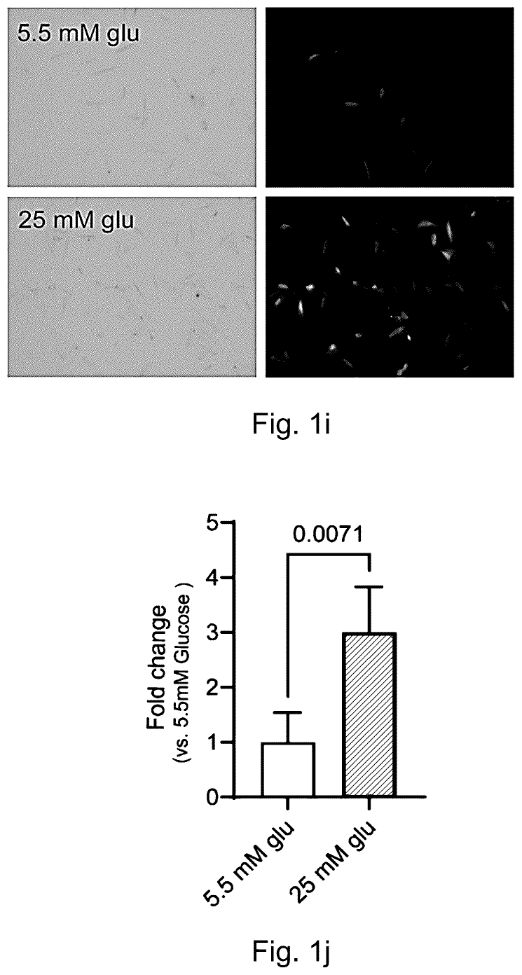

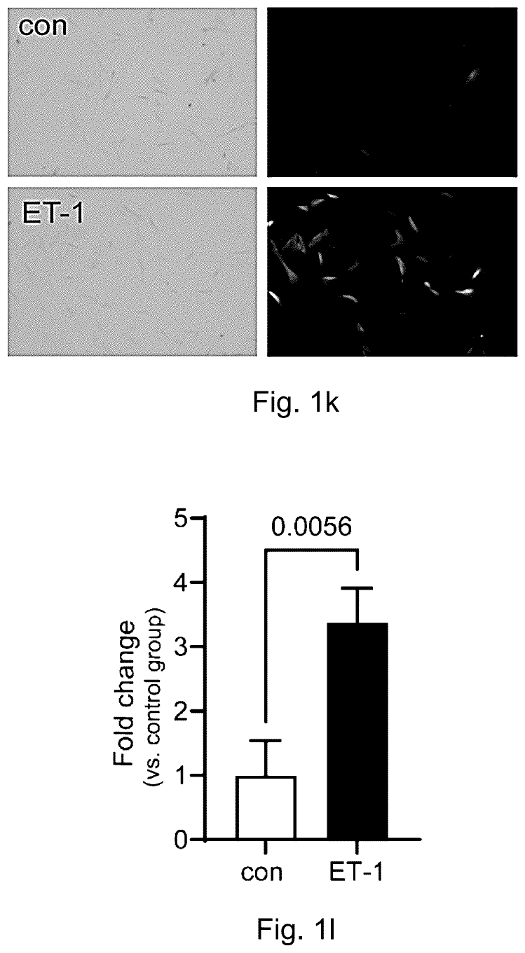

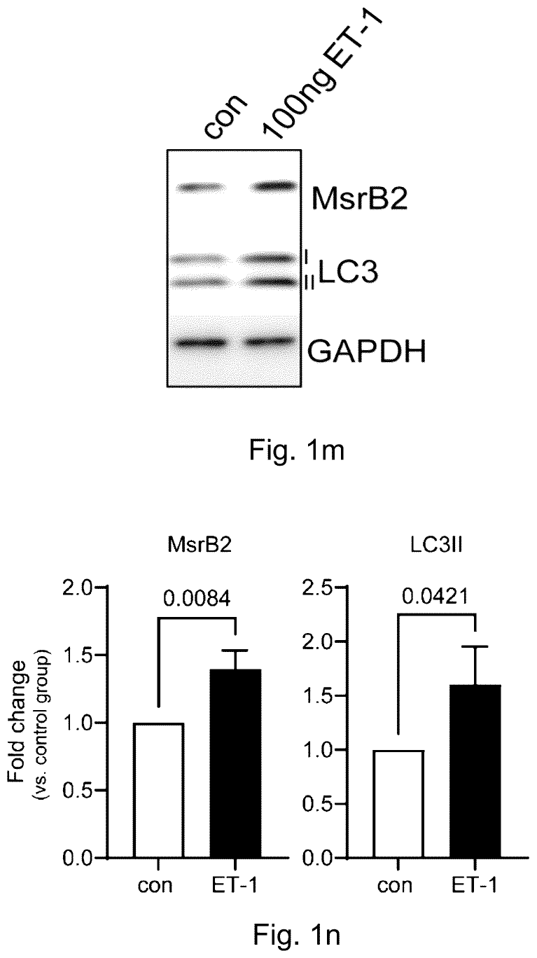

In a specific embodiment of the present invention, MsrB2 expression in the heart of a diabetic mouse model was evaluated (5 days of STZ injection followed by 12 weeks of HFD). Comparing the glucose concentrations of diabetic and normal mice, the glucose concentration of diabetic mice (DM) was approximately twice that of normal mice (FIG. 1a). Both ROS- and MsrB2-induced methionine sulfoxylation (MetO) levels were significantly increased in diabetic mice (FIGS. 1b and 1c). MsrB2 is known to increase mitophagy (selective mitochondrial autophagy) by binding to Parkin and LC3II in diabetic platelets. Consistent with the increase in MsrB2 in diabetic mouse hearts, it was confirmed that Parkin levels and the active form of LC3II were also increased (FIGS. 1d and 1e). N-acetylcysteine (NAC), a ROS inhibitor, was administered intraperitoneally to diabetic mice to determine whether ROS is involved in regulating the expression of MsrB2 and the increase in mitophagy observed in diabetic heart tissue. Mice were sacrificed 1 hour after the intraperitoneal administration. As a result, it was confirmed that MsrB2 and LCII expression increased with increasing ROS and decreased when NAC inhibited ROS (FIGS. 1f and 1g). The levels of ROS and LC3 activation in response to high glucose (HG) and endothelin-1 (ET-1, a myocardial hypertrophy inducer) treatment in H9C2 cardiac myoblasts were examined. As a result, the levels of ROS and LC3 activation were significantly increased under high glucose conditions (FIGS. 1h to 1j). Endothelin-1 treatment significantly increased MsrB2 as well as ROS and LC3 activation (FIGS. 1k to 1n). These results suggest that cardiac mitophagy is associated with the activation of ROS, MsrB2, and LC3.

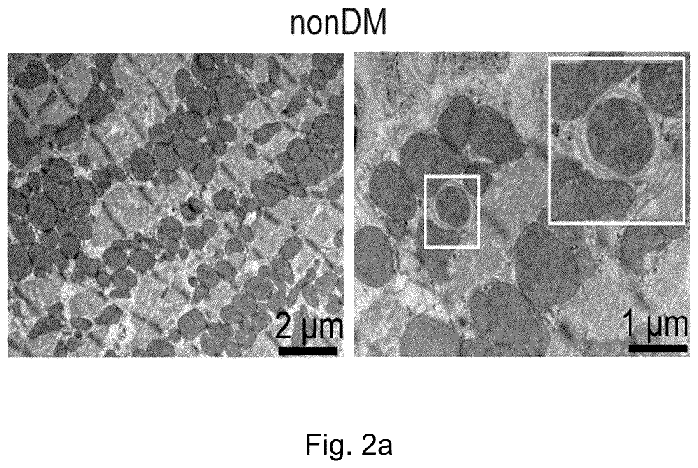

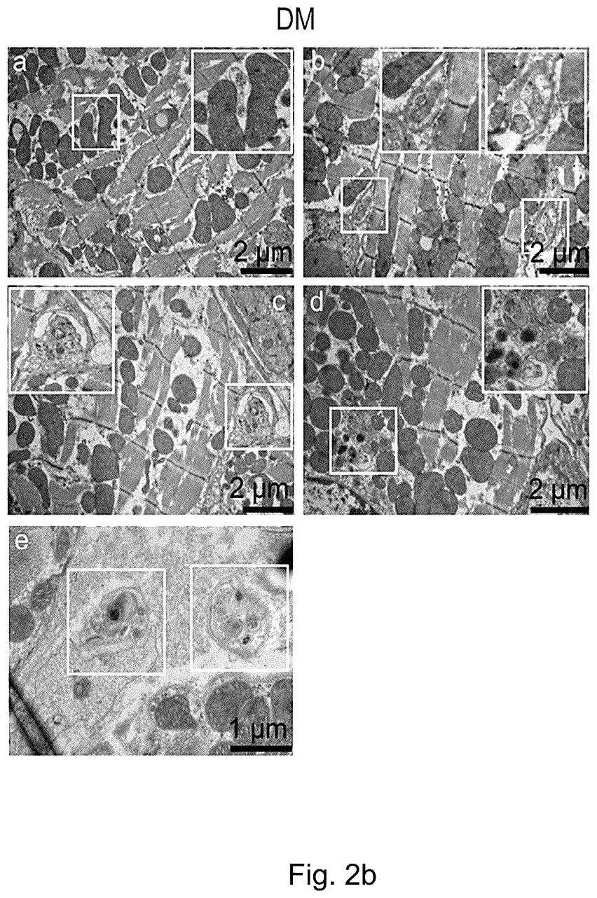

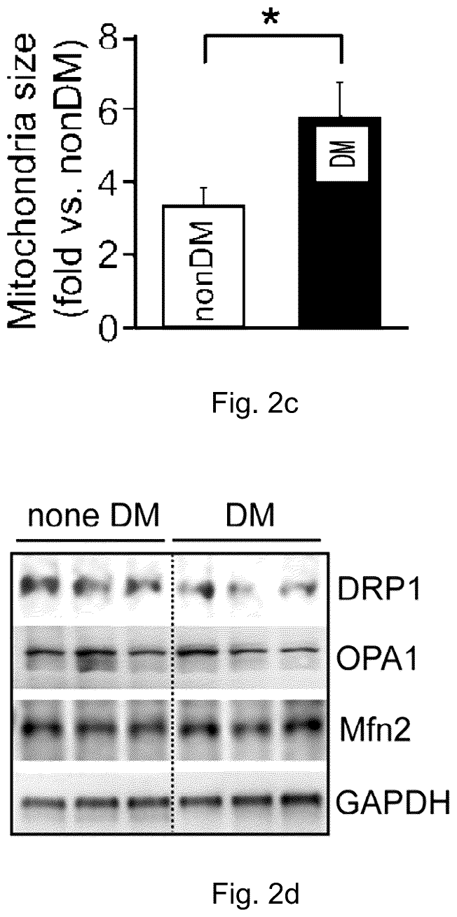

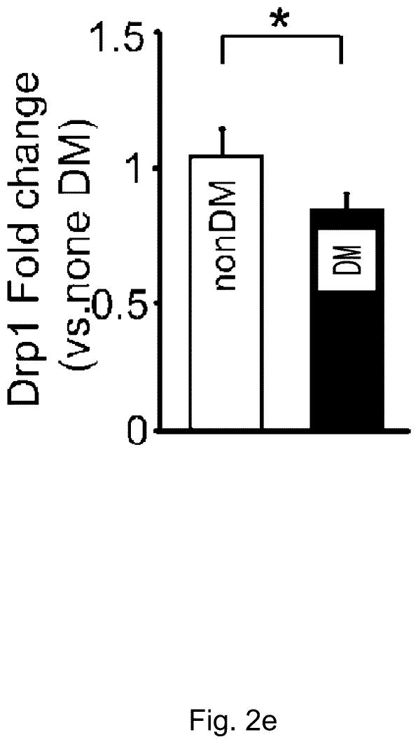

The structural integrity of mitochondria was confirmed by electron microscopy (EM) (FIGS. 2a and 2b). Myocardium was evenly distributed in normal mouse heart tissue, and mitochondria were observed to be closely aligned between contractile organelles (FIG. 2a). In contrast, diabetic hearts had irregular mitochondrial shapes (either very large or very small) and unevenly distributed myocardial architecture (FIG. 2b). It was also confirmed that the total average mitochondrial size was increased in the hearts of diabetic mice compared to normal mice (FIGS. 2b and 2c). Considering that these changes are closely related to mitochondrial fission and fusion, the present inventors performed Western blotting on diabetic and normal hearts using DRP1 as a mitochondrial fission marker and OPA1 and Mfn2 as mitochondrial fusion markers. As a result, the expression of OPA1 and Mfn2 did not change significantly, but the expression of DRP1 was confirmed to decrease in diabetic hearts compared to normal hearts (FIGS. 2d and 2e). In electron microscopy images of heart tissues of diabetic mice, vacuoles containing organelles were observed (FIG. 2b). These structures are typical autophagosomes or autolysosomes and support the idea that autophagy is increased in the diabetic heart with increased active forms of Parkin and LC3II. If these structures contain damaged mitochondria, this further supports that mitophagic autophagy is increased (FIG. 2b). Thus, it can be seen that the increased ROS (increased MetO) levels, MsrB2 induction, and mitophagy in the diabetic mouse model heart were caused by impaired mitochondrial function and biosynthesis.

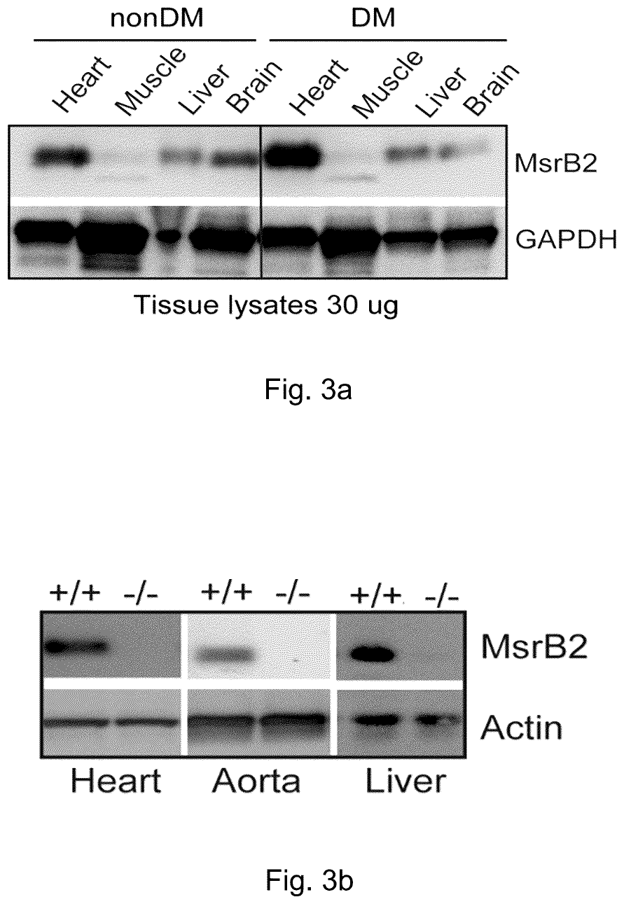

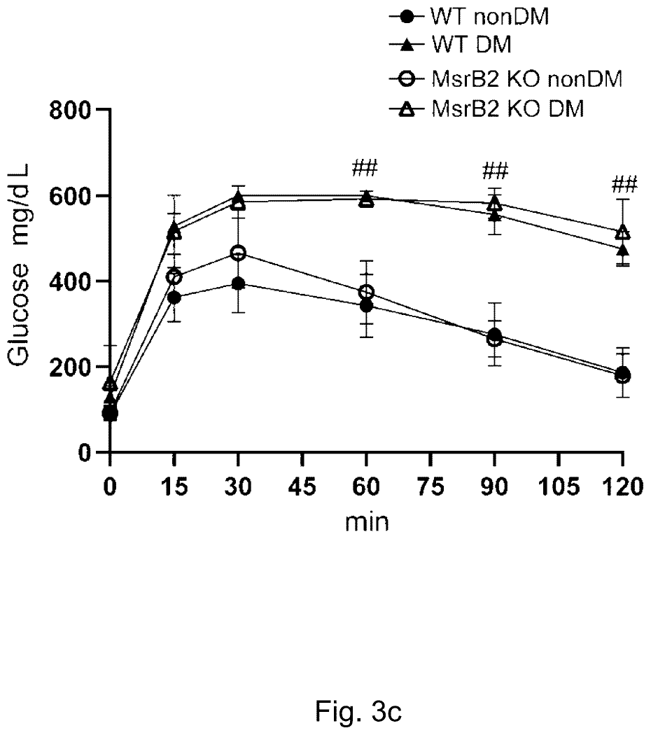

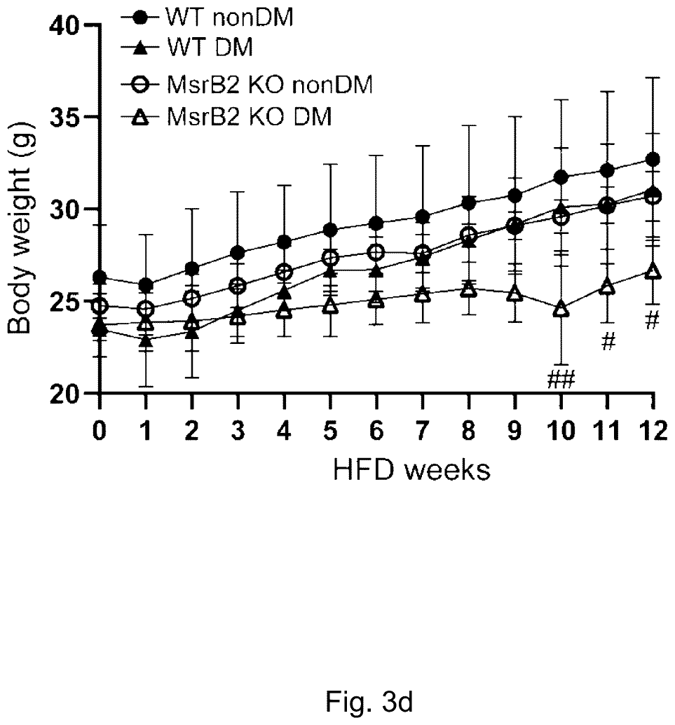

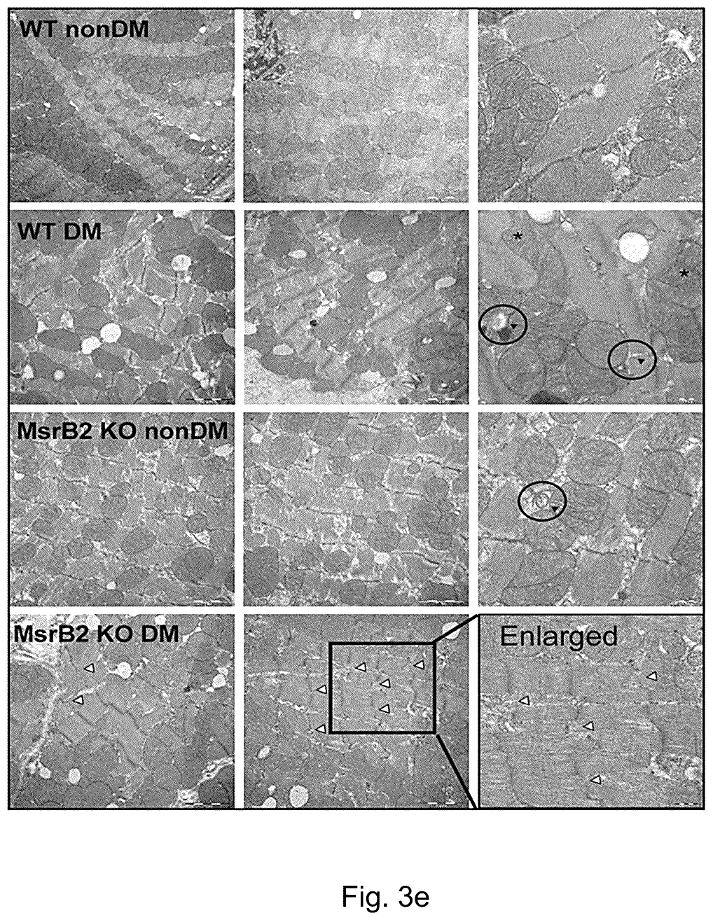

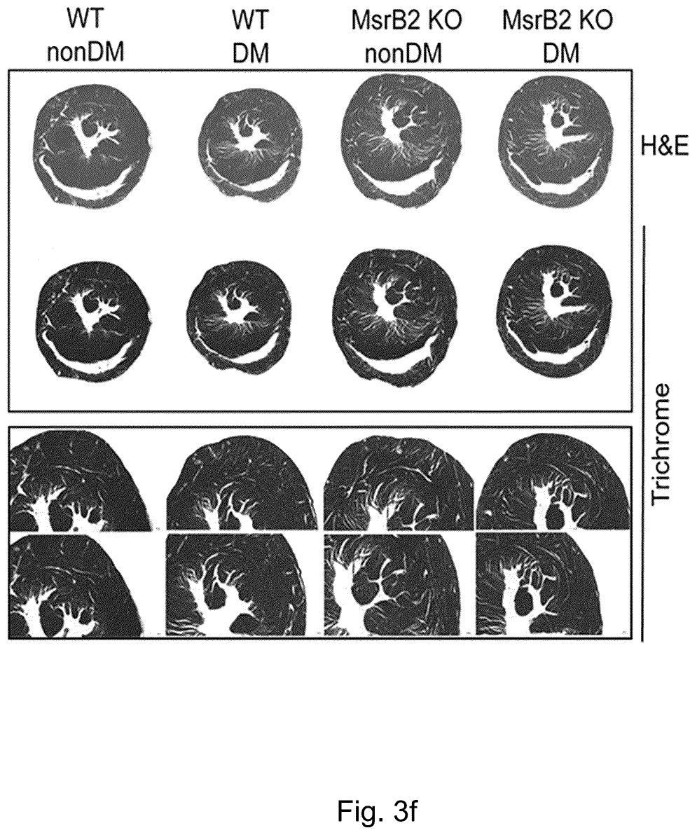

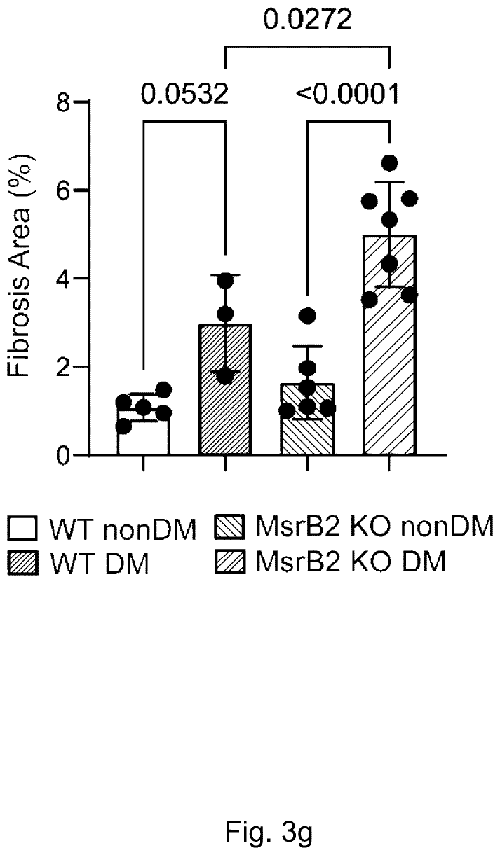

MsrB2 global knockout (KO) mice were generated, and the role of MsrB2 in diabetic heart disease and whether mitophagy is increased were investigated. As a result, it was confirmed that there was no MsrB2 expression at all in the liver, heart, and aorta tissues of the KO mice (FIGS. 3a and 3b). Five days after STZ injection into MsrB2 KO mice, a diabetic heart mouse model was created by administering HFD for 12 weeks, and a glucose tolerance test (GTT) was performed. As a result, it was confirmed that there was no difference between the knockout diabetic mouse model and normal mice (FIG. 3c). Body weight was reduced in MsrB2 knockout mice (MsrB2 KO DM) compared to wild-type diabetic mice (WT DM) and normal mice (non-DM) (FIG. 3d). These results suggest that MsrB2 KO DM mice are suitable as a lean diabetes model.

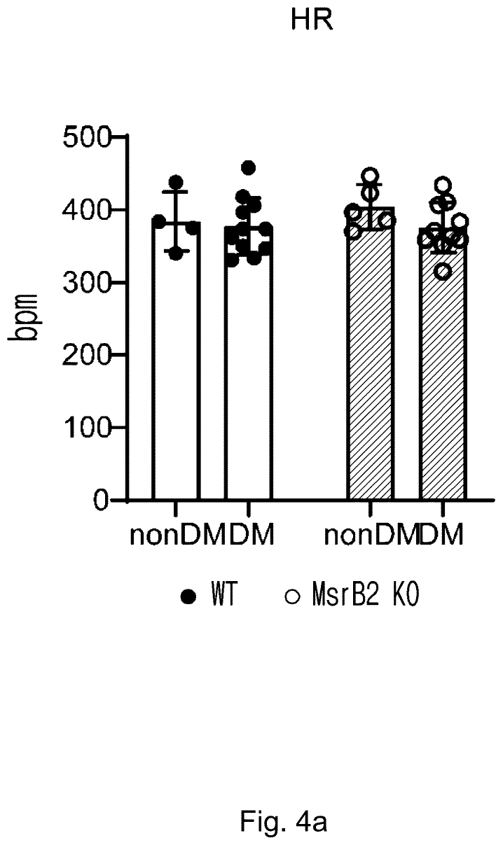

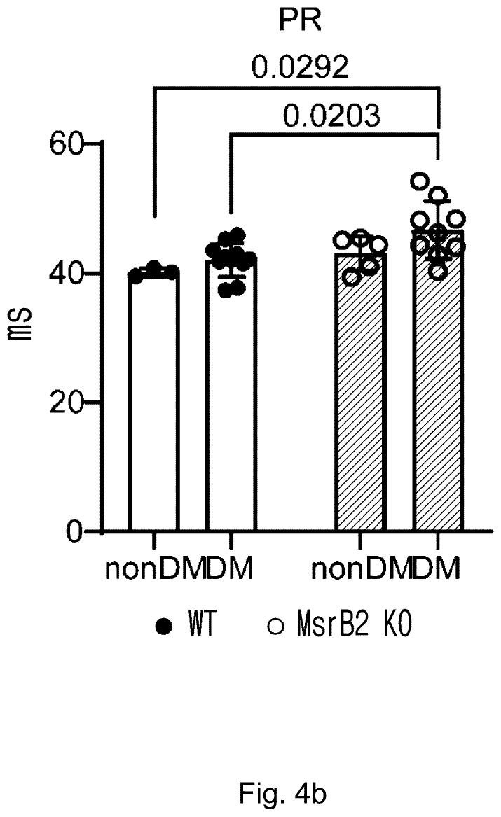

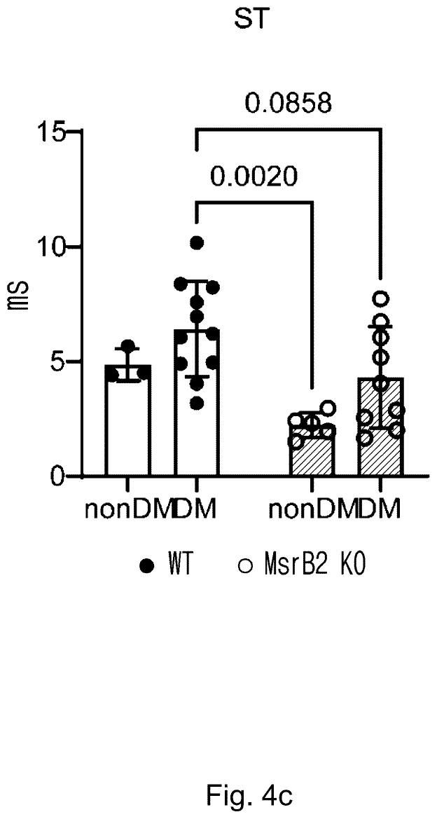

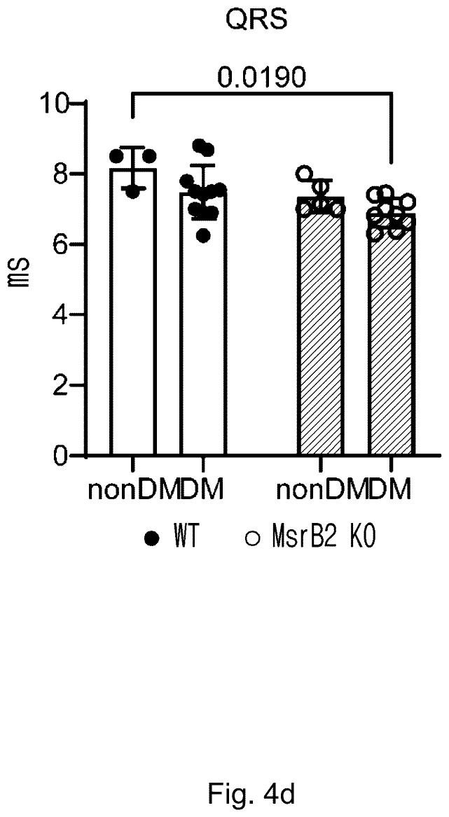

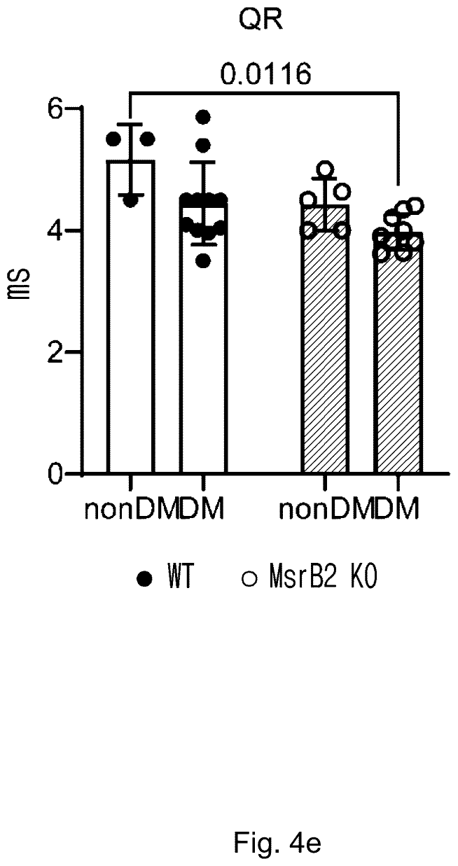

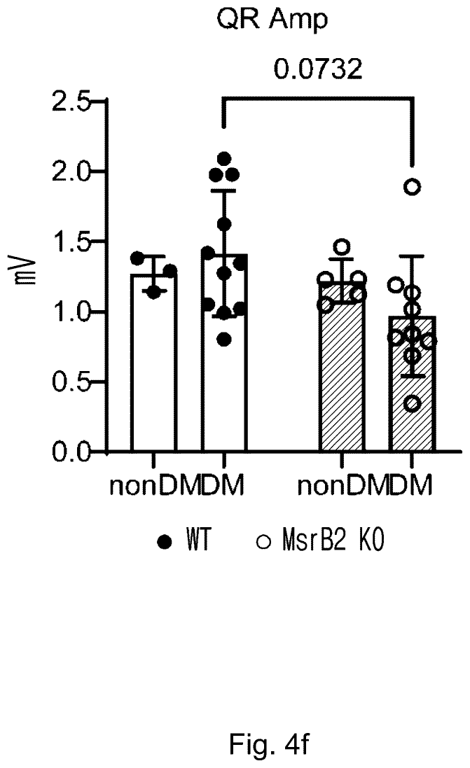

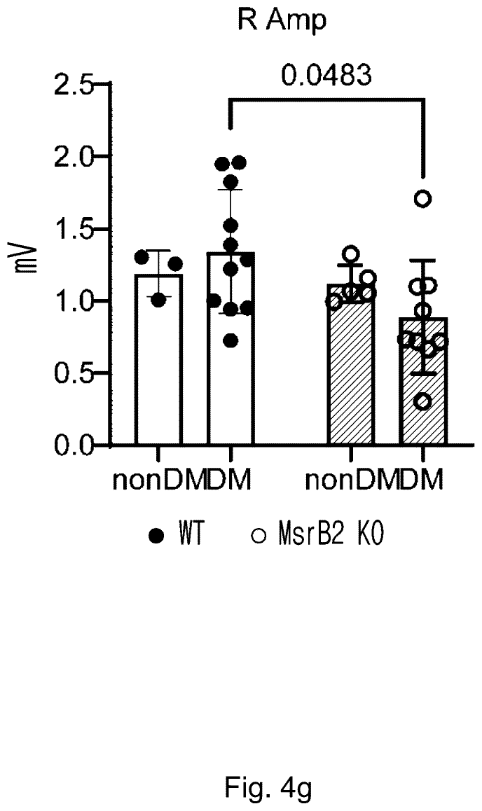

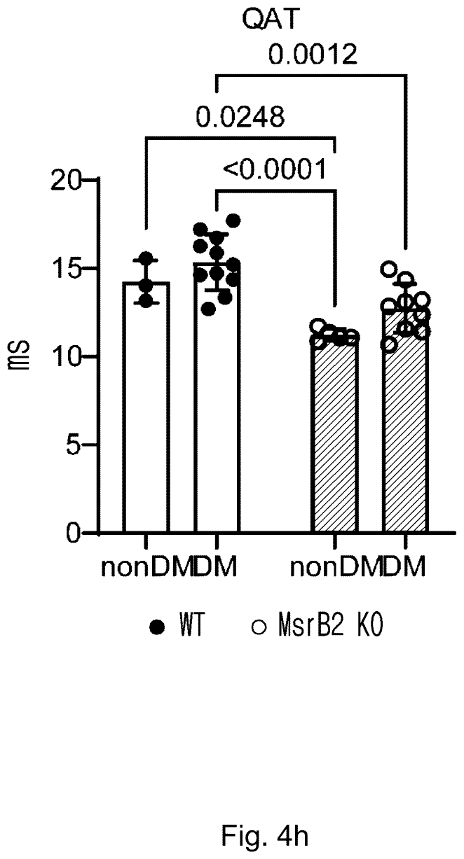

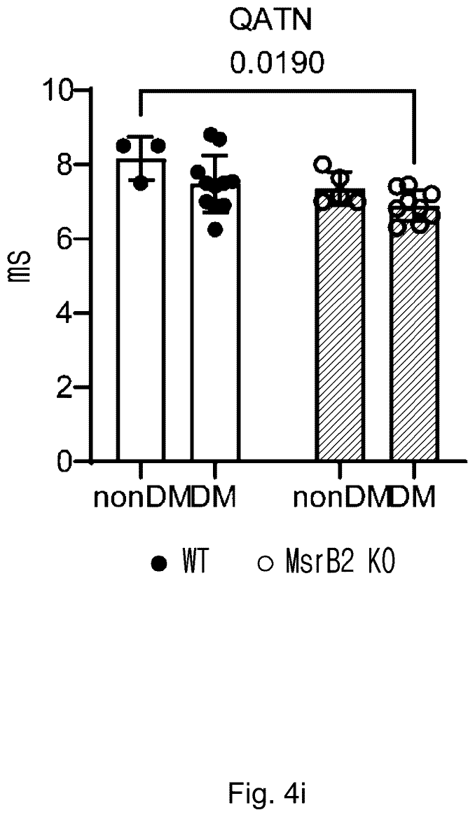

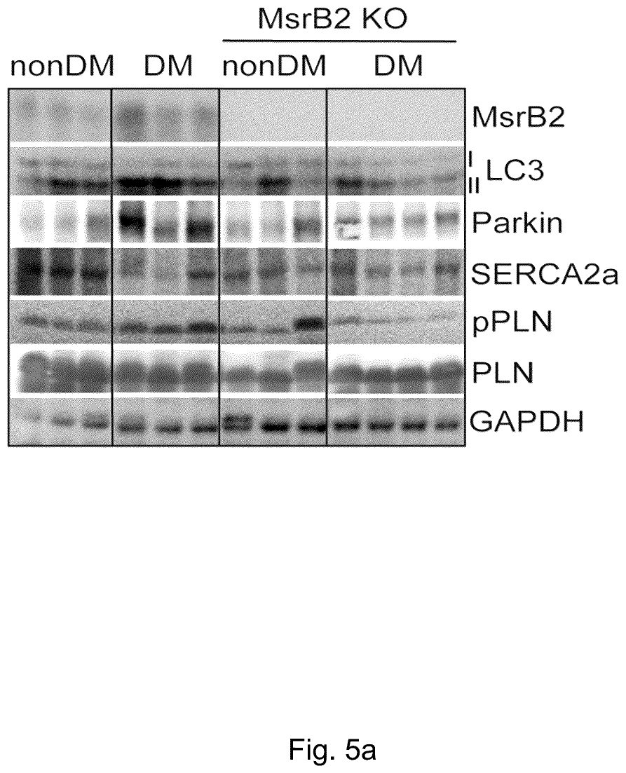

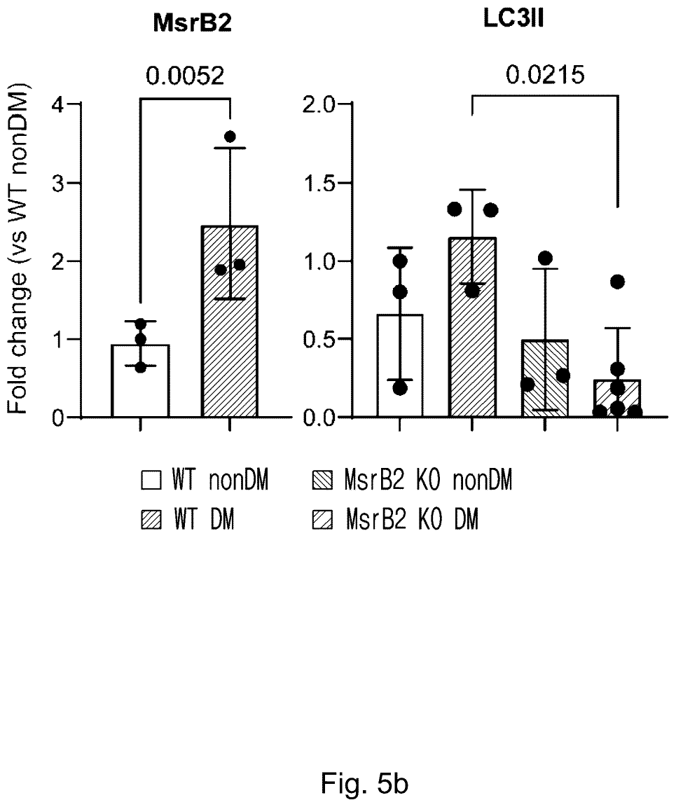

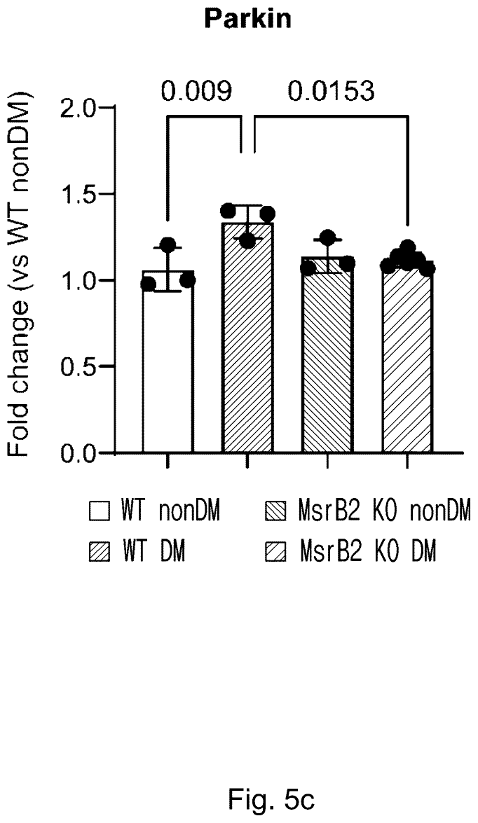

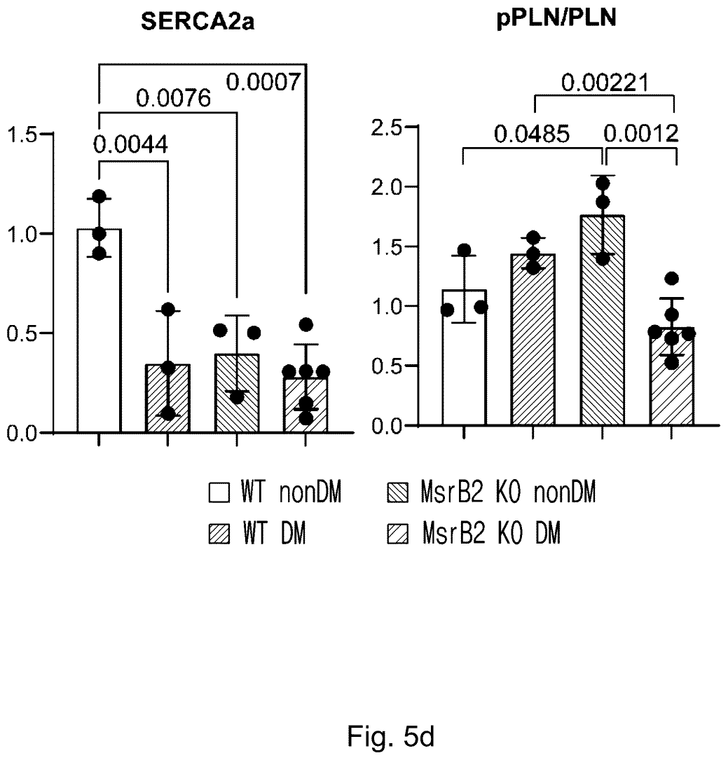

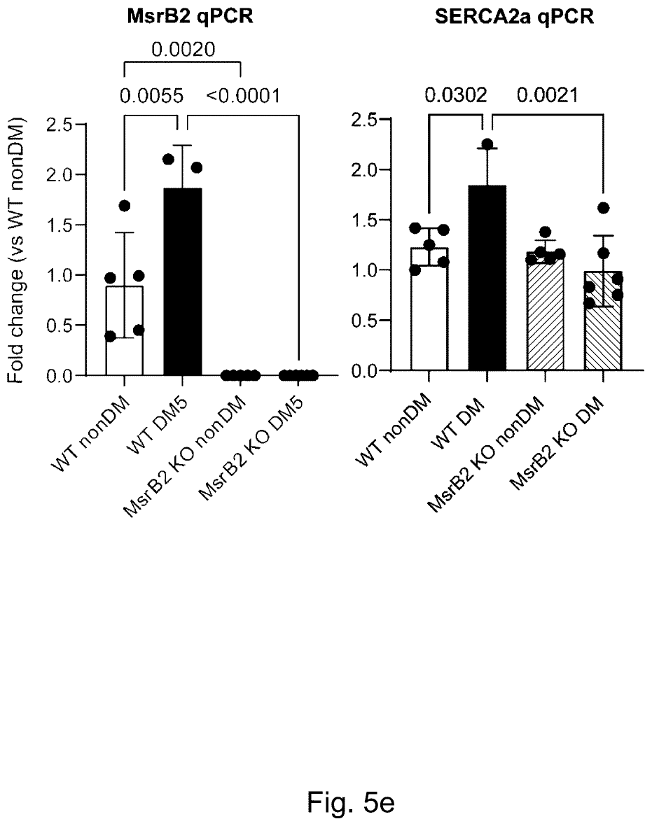

Electron microscopy images of heart tissues from a wild-type diabetic mouse model (WT DM) and an MsrB2 knockout diabetic mouse model (MsrB2 KO DM) showed mitochondrial irregularities in both conditions and elongated autophagosome membrane structures (FIG. 3e, black triangles). In particular, irregular patterns (broken and uneven) in the Z-line of the sarcomere structure were observed only in MsrB2 KO DM mice (FIG. 3e, white triangles). Histological analysis of heart tissues revealed a significant increase in cardiac fibrosis, hemorrhage, tissue fragmentation, and large vacuoles in MsrB2 KO DM mice compared to WT DM (FIGS. 3f and 3g). When examining whether there were any electrophysiological dysfunctions of the heart, the heart rate was the same in all groups (FIG. 4a). In addition, it was confirmed that the PR interval (PR interval in electrocardiogramdicates the time it takes for the electrical signal of the heart to travel from the top of the heart to the bottom) was increased in MsrB2 KO DM compared to WT DM (FIG. 4b). This may be due to atrial depolarization dysfunction in MsrB2 KO DM. It was also confirmed that the electrocardiogram parameters such as ST, QRS, QR, QR amplitude, and R amplitude were reduced (FIGS. 4c to 4g). This indicates that the ventricular depolarization function is lost in MsrB2 KO DM. Furthermore, it was confirmed that the abnormal ventricular repolarization was caused by a decrease in QAT and QATN (FIGS. 4h and 4i). Therefore, the results of electrocardiogramaxamination confirmed that abnormal electrophysiological functions were shown in MsrB2 KO DM. In addition, the expression of MsrB2 and LC3II was increased in the hearts of DM, but the active form of LC3 (LC3II) was not increased in MsrB2 KO nonDM or MsrB2 KO MS (FIGS. 5a and 5b). These results indicate that MsrB2 is an important activator of autophagy in the diabetic heart.

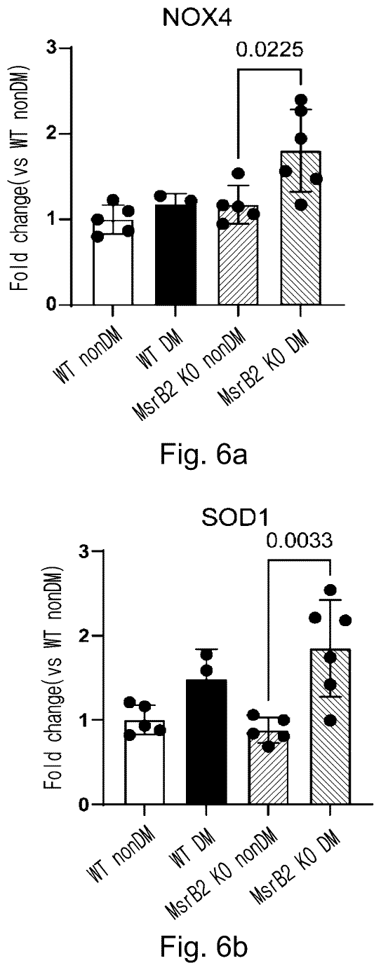

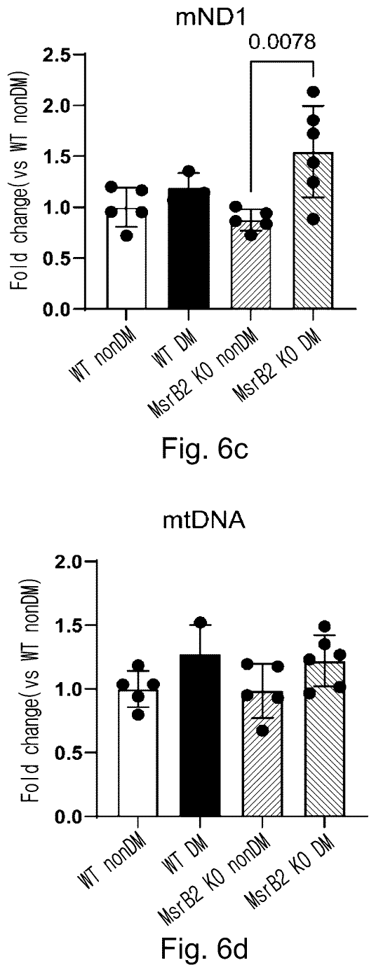

Previous studies have reported that diabetic cardiomyopathy (DCM) is caused by alterations in SERCA2a function induced by abnormal Ca²⁺ levels, and SERC2a expression levels are known to be closely related to mitochondrial quality control and mitigation of cardiac microvascular ischemia-reperfusion injury. Therefore, the present inventors confirmed the expression of SERCA2a as a marker of diabetic cardiomyopathy (DCM). As a result, the expression of SERC2a was significantly reduced in WT DM, MsrB2 KO nonDM, and MsrB2 KO DM mice, whereas phosphorylated phospholamban (PLN) was significantly reduced only in MsrB2 KO DM (FIGS. 5a to 5d). The mRNA levels of MsrB2 and SERCA2a were significantly reduced in MsrB2 KO DM mice, which were confirmed by qPCR (FIG. 5e). To determine whether the mitochondrial function was altered in MsrB2 KO DM, mRNA sequencing of the genes involved in oxidative phosphorylation and ROS production was performed. As a result, it was confirmed that the expression of all genes involved in oxidative phosphorylation and ROS production was increased in MsrB2 KO DM. However, there was no change in total mitochondrial content or mitochondrial DNA (mtDNA) (FIGS. 6a to 6d). This suggests that the accumulation of excessive ROS in the MsrB2 KO DM mouse heart contributed to the development of cardiac dysfunction, as oxidative phosphorylation and ROS production increased. Consequently, despite similar glucose tolerance in WT DM and MsrB2 KO DM mice, mitochondrial dysfunction, cardiac fibrosis, myocardial tissue abnormalities, and body weight loss in MsrB2 KO DM mice were confirmed.

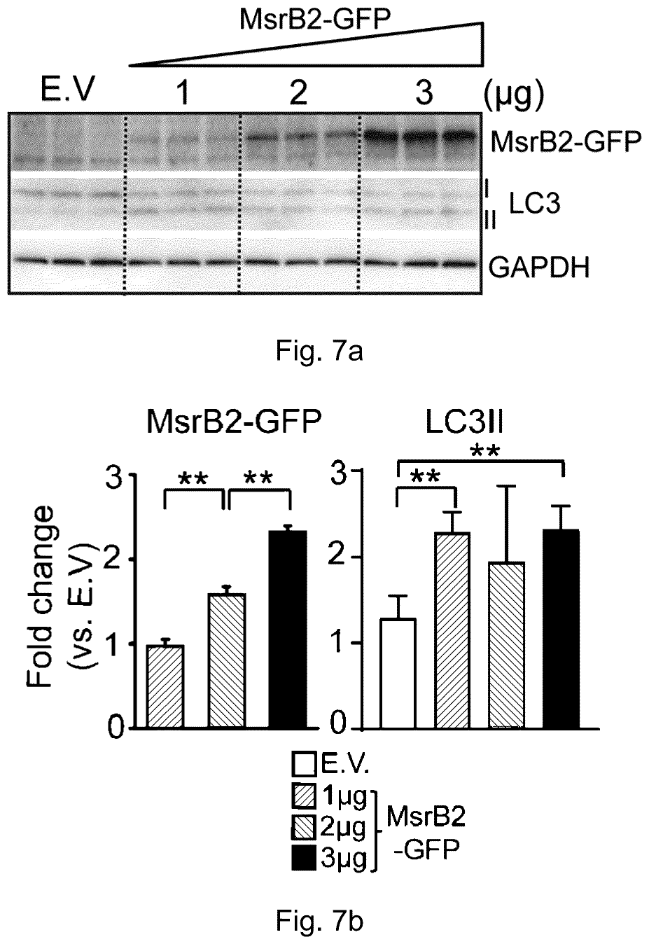

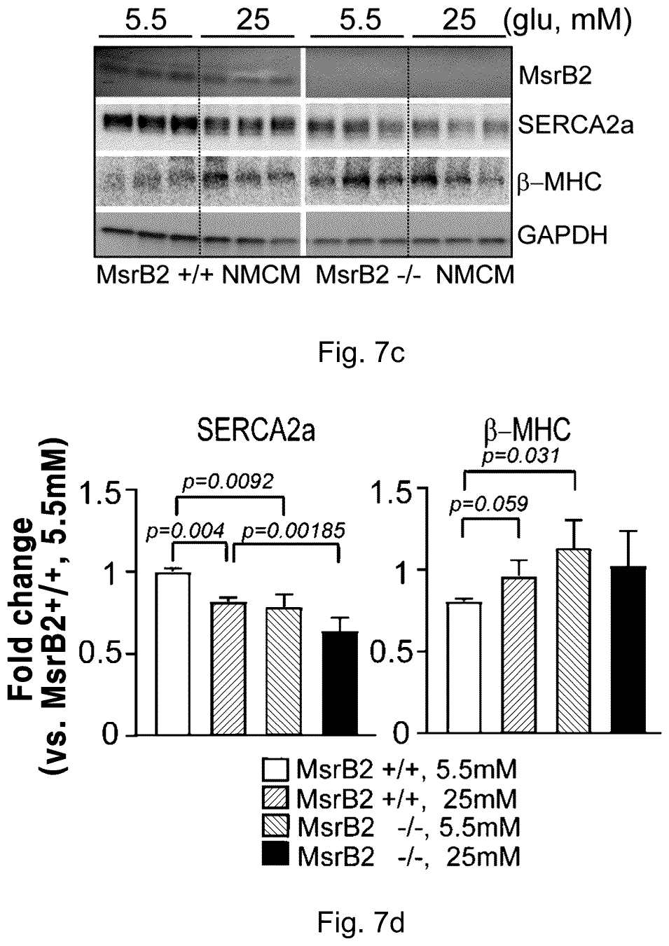

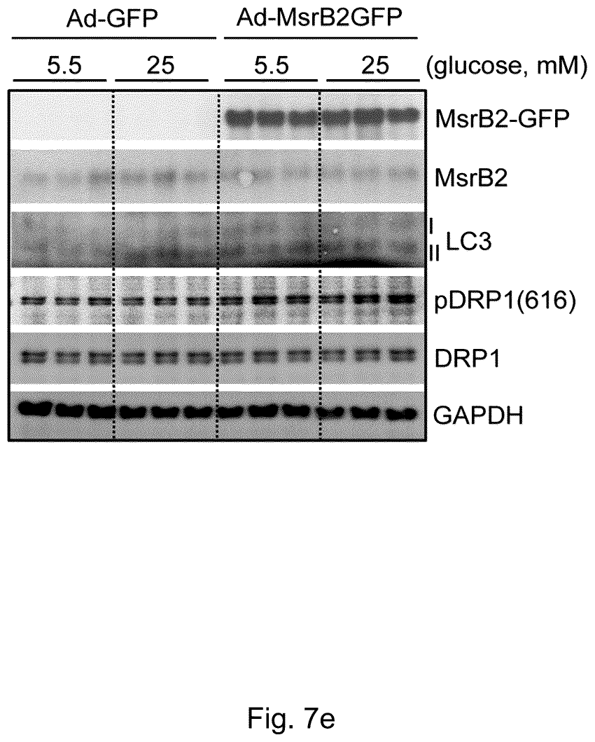

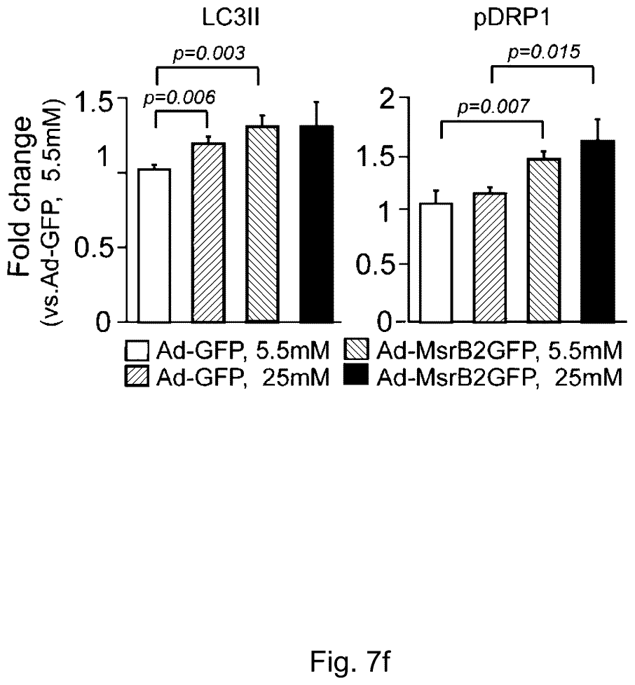

Consistent with the results in the mouse model, high glucose concentrations induced ROS and increased autophagy activity (LC3II induction) in H9C2 cells (cardiomyocytes) (FIGS. 1h to 1j). In addition, ET-1 (a cardiomyocyte hypertrophy inducer) induced ROS and increased autophagy and MsrB2 expression in H9C2 cells (FIGS. 1k to 1n). It was also confirmed that autophagy was increased in a MsrB2 concentration-dependent manner (FIGS. 7a and 7b). When mouse neonatal cardiomyocytes (MNCMs) isolated from WT mice were treated with 25 mM glucose, the expression of SERCA2a was reduced (FIGS. 7c and 7d). In MsrB2 KO MNCM, the expression of b-MHC was increased in WT and MsrB2 KO MNCM, whereas the expression of SERCA2a was significantly decreased in MsrB2 KO cells treated with 25 mM glucose compared to WT MNCM treated with 25 mM glucose (FIGS. 7c and 7d). Adenovirus-mediated overexpression of MsrB2 in mouse neonatal cardiomyocytes (MNCM) increased autophagy and DRP1 activity. These results support that depletion of MsrB2 induced a heart failure phenotype in MNCM (FIGS. 7e and 7f).

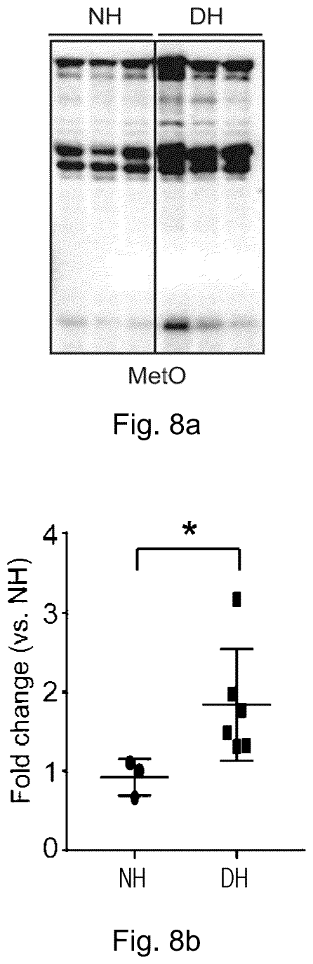

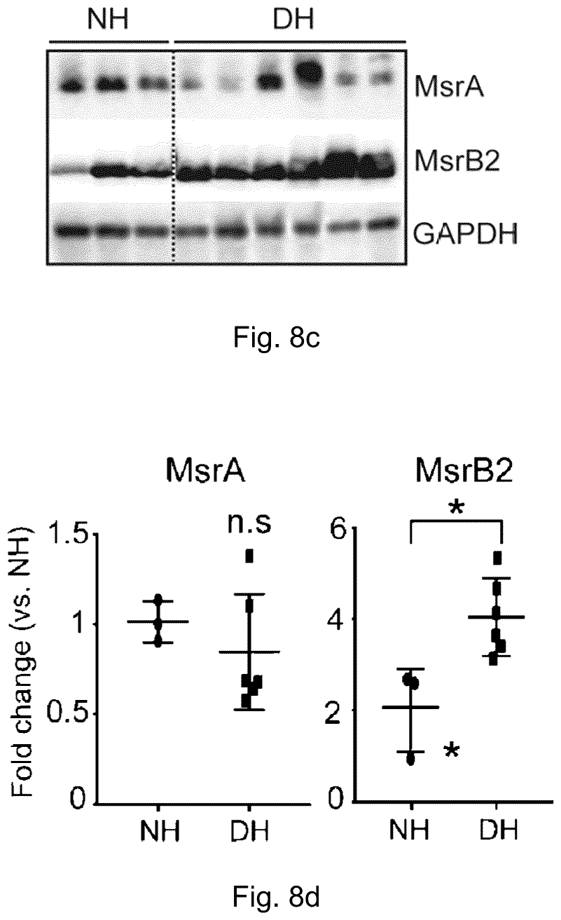

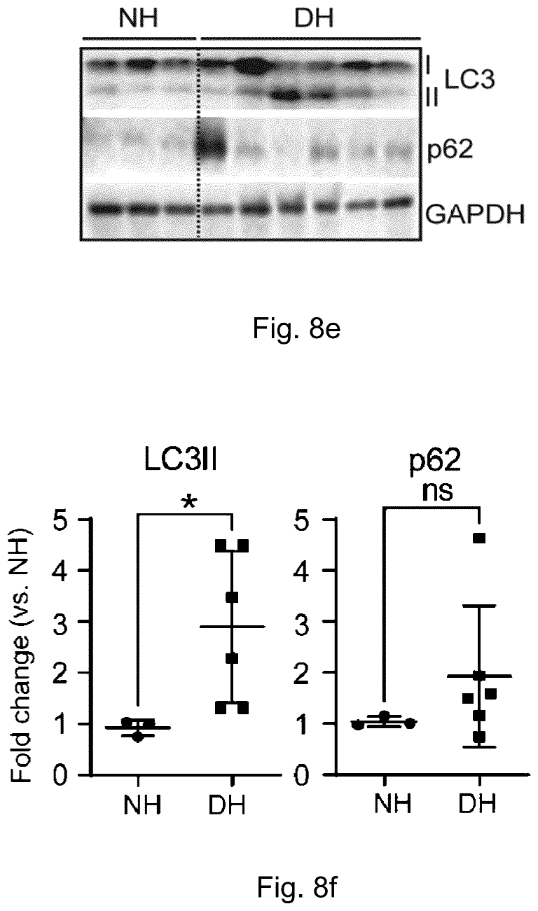

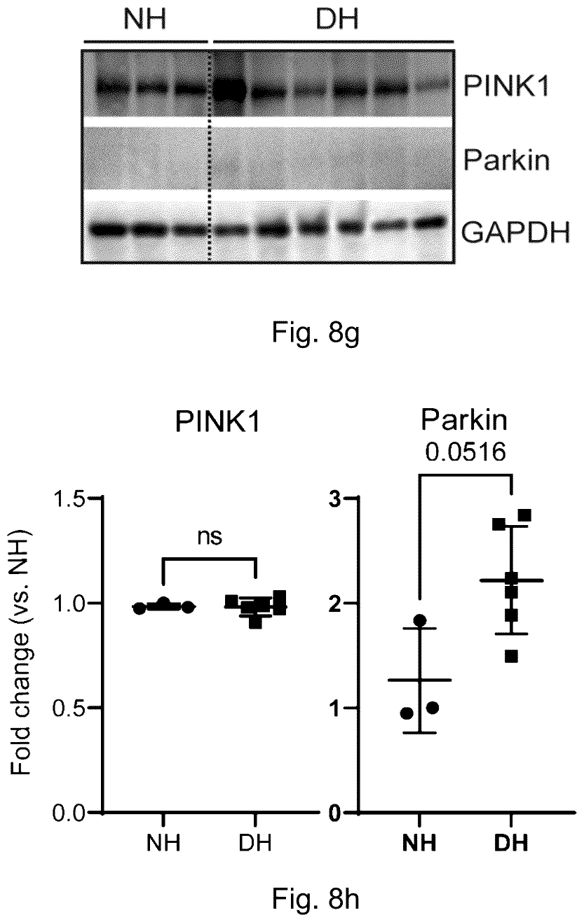

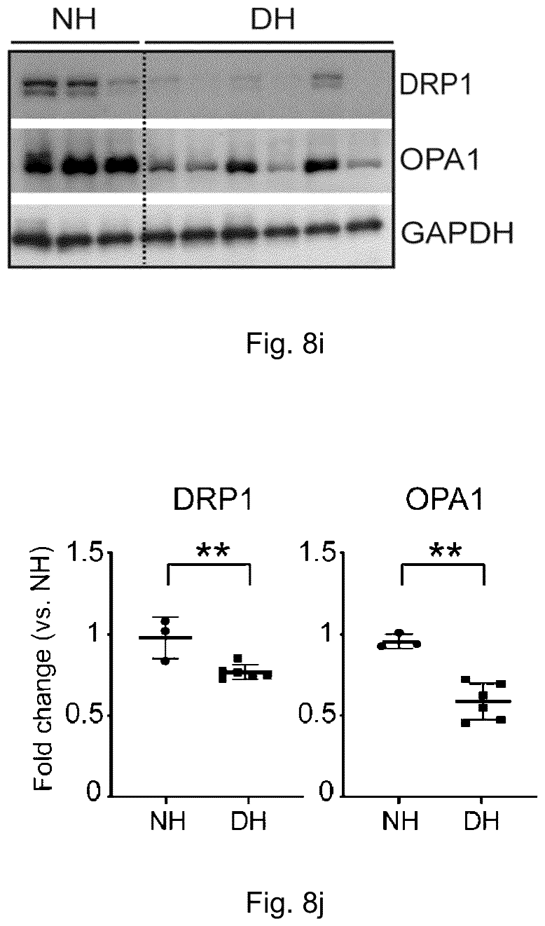

It was confirmed that MetO was increased in human diabetic heart (DH) compared to normal heart (NH) (NH (n=3); 0.92±0.23, DH (n=6) 1.83±0.70, P=0.02) (FIGS. 8a and 8b). Then, the expression levels of MsrA and MsrB were confirmed in DH (MsrA and MsrB1-B3). As a result, the expression of all Msr proteins was not significantly different compared to NH (FIGS. 8c and 8d). The LC3 II levels and Parkin expression were significantly increased, but the expression of p62 was not significantly changed compared to the HN heart (FIGS. 8e to 8h).

The expression levels of DRP1 and OPA1, the proteins involved in mitochondrial regeneration, in diabetic heart tissue were confirmed. As a result, it was confirmed that mitochondrial function was impaired (FIGS. 8i and 8j). The expression of DRP1 and OPA1 was significantly reduced in DH compared to NH, indicating a defect in mitochondrial biosynthesis in DH.

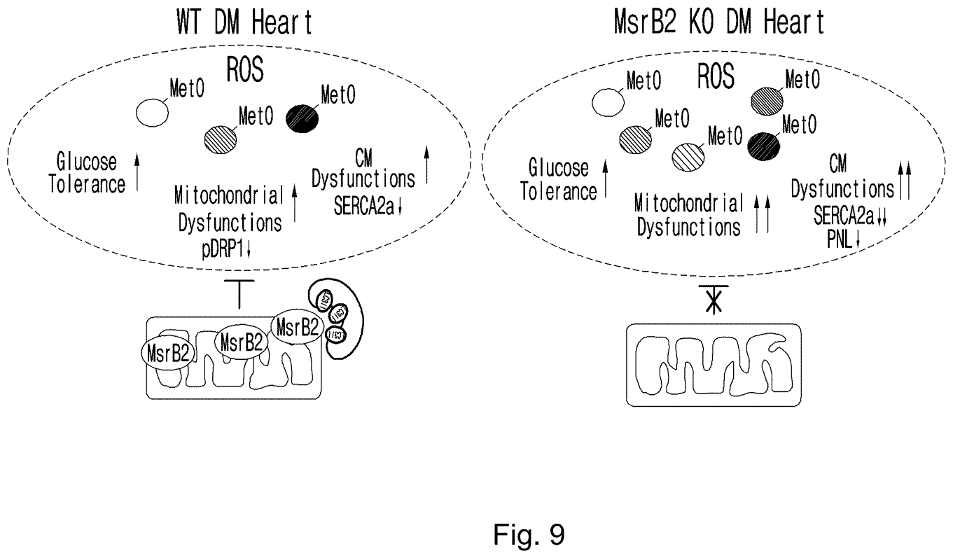

In conclusion, the present inventors confirmed that the excessive ROS (MetO indicates excessive ROS) and inactivation of autophagy in DH interfere with normal biological functions, inhibiting mitochondrial biosynthesis and leading to severe mitochondrial dysfunction. MsrB2 directly regulates the activity of LC3 and its interaction with Parkin and LC3II. In DH, the protein MetO is increased by ROS, and ROS leads to mitochondrial and myocardial dysfunction. ROS-induced MsrB2 acts to suppress this oxidative damage through LC3 activation and direct interaction with Parkin and LC3II, and induces autophagy, particularly in mitophagy. Without MsrB2, mitochondrial and myocardial damage is exacerbated, leading to severe cardiac dysfunction in diabetes (FIG. 9).

Hereinafter, the present invention will be described in detail by the following examples.

However, the following examples are only for illustrating the present invention, and the contents of the present invention are not limited thereto.

### Example 1: Methods for Preparing and Analyzing Materials

### Preparation of Human Heart Tissue

The Yale Human Investigation Committee (protocol #1,005,006,865) approved all human studies. Each subject consented to the use of all data and samples. No studies were conducted other than those approved.

### Cardiomyocyte Culture

Primary cardiomyocyte cultures were prepared from C57Bl/6 WT and MsrB2 knockout (KO) mice (Ehler et al., 2013). Ventricular tissue was enzymatically isolated, and the resulting cell suspension was centrifuged to concentrate cardiomyocytes. Then, the isolated cardiomyocytes were plated on collagen-coated culture dishes (Corning). To induce hypertrophic and diabetic cardiomyopathy, cardiomyocytes were cultured in serum-free medium for more than 4 hours and treated with 100 ng of endothelin-1 (ET-1) or 25 mM glucose for 48 hours.

### Transient Transfection

Parkin and MsrB2 ORF clones were purchased from OriGene (USA) and subcloned into vectors tagged with RFP and GFP, respectively. Cherry-LC3 was purchased from Addgene (#40,827). MsrB2-GFP, RFP-Pakin, and Cherry LC3 were transfected into H9C2 using Lipofectamine 3000 (Invitrogen, USA) according to the manufacturer's protocol. Cells were harvested in lysis buffer (50 mM Tris-HCl (pH 7.4), 150 mM NaCl, 0.25% Triton X-100 and protease inhibitor cocktail) for 48 hours for further experiments.

### Preparation of Diabetic Mouse Model

All mouse models used C57Bl/6 (WT and MsrB2 KO) mice. To generate diabetic (Diabetes Mellitus, DM) mice, 8-week-old mice were injected with streptozotocin (STZ) for 5 days and then fed a high-fat diet (HFD) for 12 weeks. Animals were housed at the Yale Animal Facility (300 George St., New Haven, CT) under the supervision of the Yale Animal Resources Center and Rita Weber (Yale CVRC Animal Facility Manager) or at the Korea NIH (LML-KCDC-11-2-26) animal facility. All experiments were performed in accordance with the appropriate guidelines and regulations according to the approved protocols 2017-11,413 and KDCA-IACUC-22-041. The fact that the mice have the same genetic background means that there are no significant differences within the groups. Therefore, all differences are directly related to processing or transformations. The experiment was validated using a different group of mice. Mice were randomly assigned to the diabetes-induced groups. The experimenter was blinded to the blood glucose levels. Further experimental validation was performed using a variety of approaches, including chemical inhibition, chemical activation, and genetic knockout, etc. The present inventors combined several random groups in different ways to reduce bias.

### Electrocardiogram (ECG) Recording

Anesthesia was performed through the induction chamber of a rodent respiratory anesthesia machine (3-4%). After induction of anesthesia, an oral inhalation anesthesia device was connected to maintain anesthesia (2%). After breathing was stabilized, the mouse was fixed in the dorsolateral position, and the ECG (−) lead was connected to the left arm, the (+) lead to the right arm, and the background lead to the right leg. ECG recording was performed for 3 minutes (IX-BIO4). After recording, the leads were removed and the subject was moved to a cage to maintain body temperature.

### Glucose Tolerance Test (GTT)

Wild-type (WT) and MsrB2 knockout (KO) mice were fed HFD once a day for 5 days for 12 weeks after STZ injection, and then food and water were withheld for 24 hours. Afterwards, 1.5 g/mg of glucose was injected intraperitoneally. To test glucose tolerance, the confirmed glucose concentration was measured over time (0, 15, 30, 60, 90, and 120 minutes).

### Western Blotting

Heart tissue lysates were obtained by homogenization in lysis buffer (50 mM Tris-HCl [pH 7.4], 150 mM NaCl, 0.25% Triton X-100, protease inhibitor cocktail [Sigma-Aldrich]). Protein lysates were loaded into each well and quantified using at least three independent replicates. Band intensities were analyzed using ImageJ analysis software (NIH), the intensity values were converted to fold changes compared to the heart complication (HC) group or the untreated group, and the fold values were used for statistical analysis.

### Electron Microscope Observation

Mouse heart tissue samples were fixed with 2% glutaraldehyde and 2% paraformaldehyde in 0.1 M sodium cacodylate (pH 7.4) for 2 hours at room temperature. The samples were washed three times with 0.1 M cacodylate buffer at room temperature, and then cells were post-fixed with 1% osmium tetroxide in 0.1 M cacodylate buffer at room temperature for 1 hour. After rinsing with cold distilled water, the tissue samples were slowly dehydrated with ethanol and propylene oxide. Samples were fixed in Embed-812 (EMS, USA) and visualized using a scanning electron microscope (Yale Biological EM Facility, New Haven, CT).

For transmission electron microscopy (TEM), heart tissues were fixed by incubation in 2% paraformaldehyde and 2.5% glutaraldehyde in 0.1 M phosphate buffer (pH 7.4) to prevent autolysis. To minimize chemical reactions before and after fixation, the grids were washed three times using the same buffer as the fixative and then post-fixed with 1% osmium tetroxide. After washing three times with deionized water, the samples were dehydrated with increasing concentrations of 30%, 50%, 70%, 80%, 90%, and 100% ethanol, and then the ethanol was replaced with propylene oxide. The tissues were then fixed in Epon812 plastic resin and polymerized at 65° C. for 48 hours. The prepared samples were cut into 70 nm thin sections using an ultramicrotome (EM-UC7, LEICA). The sections were mounted on 100-mesh copper grids and electro-stained with 4% uranyl acetate. The sections were observed with a transmission electron microscope (Libra120, Carl Zeiss, Germany) at an accelerating voltage of 120 kV.

### RNA Sequencing

Total RNA was isolated from wild-type (WT) and MsrB2 mouse hearts (non-DM and DM). RNA sequencing was performed at Bionics, Korea.

### ROS Measurement

H9C2 cells were plated in glass bottom dishes and treated with 5, 25 mM glucose or ET-1 (Sigma-Aldrich) for 48 hours. The treated cells were incubated with 1 μM H2DCFDA for 1 hour and observed using an Invitrogen EVOS M5000 cell imaging system (Invitrogen) with a 20× lens, and signal intensities were calculated using the ImageJ program.

### Immunoprecipitation (IP)

Mouse heart tissue lysates and cell lysates (after transient transfection) were mixed with specific target antibodies [for Parkin IP, 1 μg of LC3 anti-rabbit antibody (Abcam) and 2 μg of Parkin anti-goat antibody (Abcam) or 1.5 μg of Parkin anti-rabbit antibody (Abcam); for MsrB2 IP, 1 μg of MsrB2 anti-rabbit antibody (Yale); GFP-Trap beads (Chromotek, USA) and isotype IgG control including HC] and incubated overnight at 4° C. Then, 30 μL of A/G beads mixed 50:50 with 50% slurry protein A sepharose beads and 50% slurry protein G sepharose beads and washed with 50% slurry were incubated with the lysate/antibody mixture at 4° C. for 1 hour. After three more washes with lysis buffer, 1-10% lysates were used as input.

### Immunocytochemistry and Confocal Microscopy

H9C2 cells were plated in glass bottom dishes and transiently transfected with MsrB2-GFP, RFP-Parkin, and Cherry-LC3. The cells were then fixed with 4% paraformaldehyde solution (Biosesang, Korea) and observed using a Nikon Eclipse-Ti confocal microscope with a 100× oil immersion lens. Colocalization was assessed using parameters set in Volocity software (PerkinElmer, USA).

### Statistical Processing

The mouse studies were conducted blinded to glucose levels. All data were expressed as mean±standard deviation or standard error of mean±mean where appropriate. Non-parametric t-tests were performed for comparisons between the two groups. One-way or two-way ANOVA analysis of variance was performed to compare the four groups as described in separate experiments. Analysis was performed using Prism software (GraphPad Software, Inc., La Jolla, CA). P<0.05 was considered to indicate a statistically significant difference.

### Example 2: Confirmation of ROS Effect on Methionine Sulfoxylation and Methionine Sulfoxide Reductase (MsrB2) Levels in Diabetic Mouse Heart Tissue

Cardiac expression of MsrB2 was evaluated in a diabetic mouse model (5 days of STZ injection followed by 12 weeks of HFD). A comparison of glucose concentrations in diabetic and normal mice showed that diabetic (DM) mice had approximately twice the glucose concentration of normal mice (FIG. 1a). Both ROS- and MsrB2-induced methionine sulfoxylation (MetO) levels were significantly increased in diabetic mice (MetO; normal heart (n=3); 0.97±0.03, diabetic heart (n=3) 1.29±0.12, P=0.013 and MsrB2; normal heart (n=3); 1.05±0.06, diabetic heart (n=3) 1.29±0.05, P=0.009) (FIGS. 1b and 1c).

MsrB2 is known to increase mitophagy (selective mitochondria autophagy) by binding to Parkin and LC3II in diabetic platelets. Consistent with the increase of MsrB2 in diabetic mouse hearts, the Parkin levels (normal heart (n=3); 0.98±0.37, diabetic heart (n=3) 2.19±0.39, P=0.01) and active form of LC3II (normal heart (n=3); 0.85±0.28, diabetic heart (n=3) 2.16±0.38, P=0.009) were also increased (FIGS. 1d and 1e).

N-acetylcysteine (NAC), a ROS inhibitor, was administered intraperitoneally to diabetic mice to confirm whether ROS is involved in regulating the expression of MsrB2 and the increase in mitophagy observed in diabetic heart tissue. Mice were sacrificed 1 hour after the intraperitoneal administration. As a result, it was confirmed that the expression of MsrB2 and LCII was increased with the increase in ROS and decreased when NAC inhibited ROS (FIGS. 1f and 1g).

The extent of ROS and LC3 activation according to the treatment of high glucose (HG) and endothelin-1 (ET-1, a cardiac hypertrophy inducer) in H9C2 cardiac myoblasts was confirmed. As a result, under high glucose conditions, both the ROS and LC3 activation levels were significantly increased (FIGS. 1h to 1j), and when endothelin-1 was treated, not only the ROS and LC3 activation levels but also MsrB2 was significantly increased (FIGS. 1k to 1n). These results suggest that cardiac mitophagy is associated with ROS, MsrB2, and LC3 activation.

### Example 3: Confirmation of Mitochondrial Destruction in Diabetic Mouse Heart Tissue

The structural integrity of mitochondria was confirmed by electron microscopy (EM) (FIGS. 2a and 2b). Myocardium was evenly distributed in normal mouse heart tissue, and mitochondria between contractile organelles were observed to be closely aligned (FIG. 2a). In contrast, diabetic hearts had irregular mitochondrial shapes (either very large or very small) and unevenly distributed myocardial architecture (FIG. 2b). In addition, it was confirmed that the total average mitochondrial size was increased in the hearts of diabetic mice compared to those of normal mice (FIGS. 2b and 2c).

Considering that these changes are closely related to mitochondrial fission and fusion, the present inventors performed Western blotting on diabetic and normal hearts using DRP1 as a mitochondrial fission marker and OPA1 and Mfn2 as mitochondrial fusion markers. As a result, it was confirmed that there was no significant change in the expression of OPA1 and Mfn2, and that the expression of DRP1 was reduced in the diabetic heart compared to the normal group (FIGS. 2d and 2e). In electron microscopy images of heart tissues of diabetic mice, vacuoles containing organelles were observed (FIG. 2b). These structures are typical autophagosomes or autolysosomes, supporting that autophagy is increased in the diabetic heart with increased levels of the active forms of Parkin and LC3II. If these structures contain damaged mitochondria, this further supports that mitophagic autophagy is increased (FIG. 2b). Therefore, it can be seen that the increased ROS (increased MetO) levels, MsrB2 induction, and mitophagy in the hearts of diabetic mouse models were caused by impaired mitochondrial function and biosynthesis.

### Example 4: Confirmation of Availability of MsrB2 as Modulator of Diabetic Heart Complications

The association of MsrB2 function with diabetic heart has not been reported in previous literatures. Accordingly, in the present invention, the function of MsrB2 in diabetic cardiac complications was explored, and the possibility of MsrB2 being used as a diabetic cardiac complication modulator was confirmed.

The present inventors generated MsrB2 global knockout (KO) mice and investigated the role of MsrB2 in diabetic heart disease and whether mitophagy is increased. As a result, it was confirmed that the MsrB2 expression was completely absent in the liver, heart, and aorta tissues of the KO mice above (FIGS. 3a and 3b). Five days after STZ injection into MsrB2 KO mice, HFD was administered for 12 weeks to create a diabetic heart mouse model, and a glucose tolerance test (GTT) was performed. As a result, it was confirmed that there was no difference between the knockout diabetic mouse model and normal mice (FIG. 3c). Body weight was reduced in MsrB2 knockout mice (MsrB2 KO DM) compared to wild-type diabetic mice (WT DM) and normal mice (non-DM) (FIG. 3d). These results suggest that MsrB2 KO DM mice are suitable as a lean diabetes model.

Electron microscopy images of heart tissues from a wild-type diabetic mouse model (WT DM) and a MsrB2 knockout diabetic mouse model (MsrB2 KO DM) showed mitochondrial irregularities in both conditions, as well as elongated autophagosome membrane structures (FIG. 3e, black triangles). In particular, irregular patterns (broken and uneven) in the Z-line of the sarcomere structure were observed only in MsrB2 KO DM mice (FIG. 3e, white triangles).

Histological analysis of heart tissue revealed significantly increased cardiac fibrosis, hemorrhage, tissue fragmentation, and large vacuoles in MsrB2 KO DM mice compared to WT DM (FIGS. 3f and 3g).

When examined for electrophysiologic dysfunctions of the heart, the heart rate was the same in all groups (FIG. 4a). The present inventors confirmed that the PR interval (PR interval in electrocardiogramdicates the time it takes for the electrical signal of the heart to travel from the top of the heart to the bottom) was increased in MsrB2 KO DM compared to WT DM (FIG. 4b). This may be due to impaired atrial depolarization function in MsrB2 KO DM. It was also confirmed that electrocardiogram parameters such as ST, QRS, QR, QR amplitude, and R amplitude were reduced (FIGS. 4c to 4g). This indicates a loss of ventricular depolarization function in MsrB2 KO DM. In addition, the present inventors confirmed that abnormal ventricular repolarization was caused by decreased QAT and QATN (FIGS. 4h and 4i). Therefore, the electrocardiogramination results confirmed that abnormal electrophysiological functions were observed in MsrB2 KO DM. Furthermore, the expression of MsrB2 and LC3II was increased in DM hearts, but the active form of LC3 (LC3II) was not increased in MsrB2 KO nonDM or MsrB2 KO MS (FIGS. 5a and 5b). These results indicate that MsrB2 is an important activator of autophagy in the diabetic heart.

Previous studies have reported that diabetic cardiomyopathy (DCM) is caused by alterations in SERCA2a function induced by abnormal Ca2+ levels, and SERC2a expression levels are known to be closely related to mitochondrial quality control and mitigation of cardiac microvascular ischemia-reperfusion injury. Therefore, the present inventors confirmed the expression of SERCA2a as a marker of diabetic cardiomyopathy. As a result, the expression of SERC2a was significantly reduced in WT DM, MsrB2 KO nonDM and MsrB2 KO DM mice, but phosphorylated phospholamban (PLN) was significantly reduced only in MsrB2 KO DM (FIGS. 5a to 5d). The mRNA levels of MsrB2 and SERCA2a were significantly reduced in MsrB2 KO DM mice when confirmed by qPCR (FIG. 5e).

To determine whether mitochondrial function was altered in MsrB2 KO DM, mRNA sequencing of genes involved in oxidative phosphorylation and ROS production was performed. As a result, it was confirmed that the expression of all genes involved in oxidative phosphorylation and ROS production was increased in MsrB2 KO DM. However, there was no change in total mitochondrial content or mitochondrial DNA (mtDNA) (FIGS. 6a to 6d). This suggests that the increased oxidative phosphorylation and ROS production led to the accumulation of excessive ROS in the hearts of MsrB2 KO DM mice, which contributed to the development of cardiac dysfunction. In conclusion, despite similar glucose tolerance in WT DM and MsrB2 KO DM mice, mitochondrial dysfunction, cardiac fibrosis, myocardial tissue abnormalities, and body weight loss were confirmed in MsrB2 KO DM mice.

### Example 5: Confirmation of Role of MsrB2 in Hyperglycemic Condition

Consistent with the results in the mouse model, high glucose concentrations induced ROS and increased autophagy activity (LC3II induction) in H9C2 cells (cardiomyocytes) (FIGS. 1h to 1j). In addition, ET-1 (a cardiomyocyte hypertrophy inducer) induced ROS and increased autophagy and MsrB2 expression in H9C2 cells (FIGS. 1k to 1n). Autophagy was confirmed to increase in a MsrB2 concentration-dependent manner (FIGS. 7a and 7b). Mouse neonatal cardoimyocytes (MNCM) isolated from WT mice were treated with 25 mM glucose, and it was confirmed that the expression of SERCA2a was reduced (FIGS. 7c and 7d). In MsrB2 KO MNCM, b-MHC expression was increased in both WT and MsrB2 KO MNCM, whereas SERCA2a expression was significantly reduced in MsrB2 KO cells treated with 25 mM glucose compared to WT MNCM treated with 25 mM glucose (FIGS. 7c and 7d).

Adenovirus-mediated overexpression of MsrB2 in mouse neonatal cardiomyocytes (MNCM) resulted in increased autophagy and DRP1 activity. These results support that depletion of MsrB2 induced the heart failure phenotype in MNCM (FIGS. 7e and 7f).

### Example 6: Confirmation of Increased MetO in Human Diabetic Heart Tissue

In human diabetic heart (DH), MetO was confirmed to be increased compared to normal heart (NH) (NH (n=3); 0.92±0.23, DH (n=6) 1.83±0.70, P=0.02) (FIGS. 8a and 8b). The present inventors then checked the expression levels of MsrA and MsrB in DH (MsrA and MsrB1-B3) and found that the expression of all Msr proteins was not significantly different compared to NH (FIGS. 8c and 8d). The LC3 II levels and Parkin expression were significantly increased, but the expression of p62 was not significantly changed compared to HN hearts (FIGS. 8e to 8h).

By examining the expression levels of DRP1 and OPA1, the proteins involved in mitochondrial regeneration, in diabetic heart tissue, it was confirmed that mitochondrial function was impaired (FIGS. 8i and 8j). The expression of DRP1 and OPA1 was significantly reduced in DH compared to NH, indicating a defect in mitochondrial biosynthesis in DH.

In conclusion, the present inventors confirmed that excessive ROS (MetO indicates excessive ROS) and inactivation of autophagy in DH interfere with normal biological functions, inhibiting mitochondrial biosynthesis and leading to severe mitochondrial dysfunction. MsrB2 directly regulates the activity of LC3 and its interaction with Parkin and LC3II. In DH, the protein MetO is increased by ROS, and ROS lead to mitochondrial and myocardial dysfunction. ROS-induced MsrB2 acts to suppress this oxidative damage through LC3 activation and direct interaction with Parkin and LC3II, and induces autophagy, particularly in mitophagy. Absence of MsrB2 may worsen mitochondrial and myocardial damage, leading to severe cardiac dysfunction in diabetes (FIG. 9)

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The use of the terms “a” and “an” and “the” and “at least one” and similar referents in the context of describing the invention (especially in the context of the following claims) are to be construed to cover both the singular and the plural, unless otherwise indicated herein or clearly contradicted by context.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

What is claimed is:

1. A method for preventing or treating diabetic cardiomyopathy, comprising administering a pharmaceutical composition comprising an MsrB2 activator as an active ingredient.

2. The method for preventing or treating diabetic cardiomyopathy according to claim 1, wherein the MsrB2 activator increases or upregulates the MsrB2 gene expression.

3. The method for preventing or treating diabetic cardiomyopathy according to claim 1, wherein the MsrB2 activator increases the amount of MsrB2 protein expression.

4. The method for preventing or treating diabetic cardiomyopathy according to claim 1, wherein the diabetes is type II diabetes.

5. The method for preventing or treating diabetic cardiomyopathy according to claim 1, wherein the MsrB2 activator i) inhibits oxidative damage by increasing LC3II and Parkin expression, ii) activates energy metabolism by reducing ROS production, iii) increases the expression of SERCA2a and phospholamban, and iv) improves cardiac function by increasing mitochondrial regeneration and biosynthesis in heart tissue.

6. A method for determining diabetic cardiomyopathy in a subject comprising:
1) preparing cardiomyocytes isolated from the subject;
2) measuring the expression level of MsrB2 protein or MsrB2 mRNA in the cardiomyocytes obtained in step 1);
3) comparing the expression level of the MsrB2 protein or MsrB2 mRNA obtained in step 2) and, when the expression level in the cardiomyocytes isolated from the subject is lower than that in cardiomyocytes isolated from a normal control group, determining diabetic cardiomyopathy in the subject.

7. The method according to claim 6, wherein the subject is a mammal that is at risk of developing diabetic cardiomyopathy or has developed diabetic cardiomyopathy.

8. The method according to claim 7, wherein the mammal is mice, livestock, or humans.

9. The method according to claim 6, wherein the cardiomyocytes isolated from the subject are mouse cardiomyocytes (H9C2).

10. The method according to claim 9, wherein the cardiomyocytes are cardiomyocytes isolated and identified from the heart of a mouse within 3 days after birth.

11. A screening method for an substance having preventive or therapeutic activity against diabetic cardiomyopathy, comprising the following steps:
1) a step of preparing cardiomyocytes isolated from a subject;
2) a step of measuring the ROS activity and MsrB2mRNA expression levels before and after treating the prepared cardiomyocytes with candidate substances; and
3) a step of determining a candidate substance that increases the MsrB2 mRNA expression and reduces the ROS activity compared to before treatment with the candidate substance, as an substance with therapeutic activity for diabetic cardiomyopathy.

12. The screening method for an effective substance having preventive or therapeutic activity against diabetic cardiomyopathy according to claim 11, wherein the diabetes is type II diabetes.
